(19) United States
(12) Patent Application Publication
(10) Pub. No.: US 2025/0213680 A1
(43) Pub. Date: Jul. 3, 2025

(54) LIPID NANOPARTICLE COMPOSITION COMPRISING GALLIC ACID DERIVATIVE LIPID AND USE THEREOF

(71) Applicants: KOREA INSTITUTE OF SCIENCE AND TECHNOLOGY, Seoul (KR); THE CATHOLIC UNIVERSITY OF KOREA INDUSTRY-ACADEMIC COOPERATION FOUNDATION, Seoul (KR)

(72) Inventors: Eun-Kyoung Bang, Seoul (KR); Gyochang KEUM, Seoul (KR); Jae- Hwan Nam, Bucheon-si (KR); Sung Pil Kwon, Seoul (KR); Soyeon Yoo, Seoul (KR); Muhammad Faisal, Seoul (KR); Seohyeon Bae, Gimhae-si (KR)

(73) Assignees: KOREA INSTITUTE OF SCIENCE AND TECHNOLOGY, Seoul (KR); THE CATHOLIC UNIVERSITY OF KOREA INDUSTRY-ACADEMIC COOPERATION FOUNDATION, Seoul (KR)

(21) Appl. No.: 19/000,614

(22) Filed: Dec. 23, 2024

(30) Foreign Application Priority Data
Dec. 28, 2023 (KR) 10-2023-0195342

(51) Int. Cl. A61K 39/39; A61K 9/1271; A61K 9/51; A61K 39/00; A61K 47/26; A61P 37/04; C12N 15/88

(52) U.S. Cl. CPC A61K 39/39; A61K 9/1271; A61K 9/5123; A61K 47/26; A61P 37/04; C12N 15/88; A61K 2039/55555

(57) ABSTRACT

The present disclosure relates to a lipid nanoparticle composition including a gallic acid derivative lipid and a use thereof, wherein it is possible to improve delivery efficiency and immune effect by changing some of the compositions of lipid nanoparticles using a bio-friendly gallic acid derivative lipid and, in particular, if a gallic acid derivative replaces a certain ratio of an ionizable lipid in a molar composition ratio, it is possible to mitigate toxicity generated by the ionizable lipid, so as to be utilized for drug delivery more safely in vivo.

### CROSS-REFERENCE TO RELATED APPLICATION

This application claims the benefit of Korean Patent Application No. 10-2023-0195342 filed on Dec. 28, 2023, in the Korean Intellectual Property Office, the entire disclosure of which is incorporated herein by reference for all purposes.

### BACKGROUND OF THE INVENTION

The present disclosure relates to a lipid nanoparticle composition for stable delivery of nucleic acid drugs and immunity enhancement and, more specifically, to a lipid nanoparticle composition including a gallic acid derivative lipid and a use thereof.

An mRNA-based vaccine prevents a disease or alleviates the severity thereof by injecting mRNA encoding a specific antigen of the disease into the body and utilizing the immune response to an antigen expressed in vivo. The mRNA-based vaccine can be rapidly mass-produced in a short period of time if the antigen for the disease is clearly identified, which led to emergency approval by the FDA during the COVID-19 pandemic to have mRNA-based vaccines used worldwide.

Although the mRNA-based vaccine has the advantage of being able to be developed quickly, it is rapidly degraded by RNase in vivo, such that a delivery platform is necessarily required to allow it to be stably delivered into cells. mRNA-based vaccines from Pfizer and Moderna used in the COVID-19 pandemic employed lipid nanoparticles as a delivery platform with proven efficiency which had already been utilized in 2018 for delivery of other RNA-based therapeutics. Lipid nanoparticles are mainly manufactured by mixing ionizable lipids, phospholipids, cholesterol, and PEG-lipids in an optimal ratio, and they stabilized the mRNA and increased the efficiency of delivery into the cell compared to injection of the mRNA itself.

However, the ionizable lipid, which plays a role in increasing the embedding efficiency of mRNA carriers, is often cytotoxic as it is, which may cause side effects when excessive doses are applied. In addition, the lipid nanoparticles currently developed have a problem in that the efficiency of producing neutralizing antibodies which help defend cells in the body when infected is still not high.

### SUMMARY OF THE INVENTION

An object of the present disclosure is to provide a lipid nanoparticle capable of reducing side effects caused by an ionizable lipid and increasing a delivery efficiency and a neutralizing antibody production efficiency by using a bio-friendly material.

Another object of the present disclosure is to provide a drug delivery and immune enhancement use of the lipid nanoparticle.

To achieve the above objects, the present disclosure provides a lipid nanoparticle composition including a compound represented by the following Chemical Formula 1, a derivative thereof, or a pharmaceutically acceptable salt thereof as an active ingredient:

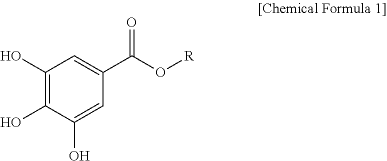

wherein, in the Chemical Formula 1, R may be a linear, saturated or unsaturated hydrocarbon with 3 to 22 carbon atoms.

The present disclosure provides a lipid nanoparticle composition including the compound represented by Chemical Formula 1, a derivative thereof, or a pharmaceutically acceptable salt thereof; an ionizable lipid; a helper lipid; and a polyethylene glycol (PEG)-modified lipid.

The present disclosure provides a composition for drug delivery, including the lipid nanoparticle composition; and a therapeutic or prophylactic agent.

In addition, the present disclosure provides an immunoenhancing composition, including the lipid nanoparticle composition.

A lipid nanoparticle according to the present disclosure includes a bio-friendly gallic acid derivative and thus may improve the immune effect by replacing a part of an ionizable lipid, a helper lipid, and a structure-maintaining lipid and, in particular, it is expected that, when a gallic acid derivative replaces a certain ratio of ionizable lipid in a molar component ratio, the toxicity caused by the ionizable lipid may be mitigated.

In addition, when mRNA was delivered using the lipid nanoparticle containing the gallic acid derivative in vivo, the expression efficiency of proteins encoded in the mRNA as well as immunoglobulin G (IgG) were enhanced compared to those not containing the gallic acid derivative, and MCP-1 concentration was lowered, such that it is anticipated to mitigate the toxicity.

### BRIEF DESCRIPTION OF THE DRAWINGS

FIG. 1 shows a result of analyzing a protein expression efficiency by measuring an amount of R/L expressed 6 hours after administering lipid nanoparticles (LNP 201a, 201b, 201c, 201d), which is in a composition having a gallic acid derivative that includes Renilla Luciferase (R/L) mRNA, and positive control lipid nanoparticles (LNP 126) into the ears of ICR mice via intradermal injection (ID.).

FIG. 2 shows a result of analyzing a protein expression efficiency by measuring an amount of R/L expressed 6 hours after administering lipid nanoparticles (LNP 218, 219, 220, 221, 284, 285, 286, 287, 297, 298, 299, 300), which is in a composition having a gallic acid derivative that includes Renilla Luciferase (R/L) mRNA, and positive control lipid nanoparticles (LNP 126, 128) into the ears of ICR mice via I.D.

FIG. 3 shows a result of analyzing a protein expression efficiency by measuring an amount of R/L expressed 6 hours after administering lipid nanoparticles (LNP 222, 223, 288, 289, 301, 302, 316, 317, 318, 319, 320, 321, 343, 344, 347, 348), which is in a composition having a gallic acid derivative that includes Renilla Luciferase (R/L) mRNA, and positive control lipid nanoparticles (LNP 126) into the ears of ICR mice via I.D.

FIG. 4 shows a result of analyzing a protein expression efficiency by measuring an amount of R/L expressed 6 hours after administering lipid nanoparticles (LNP 339, 340, 341, 342), which is in a composition having a gallic acid derivative that includes Renilla Luciferase (R/L) mRNA, and positive control lipid nanoparticles (LNP 126) into the ears of ICR mice via I.D.

FIG. 5 shows a result of analyzing a protein expression efficiency by measuring an amount of R/L expressed 6 hours after administering lipid nanoparticles (LNP 349, 350, 351, 352, 353, 354, 355, 356, 357, 358, 359, 360, 361, 362, 363, 364), which is in a composition having a gallic acid derivative that includes Renilla Luciferase (R/L) mRNA, and positive control lipid nanoparticles (LNP 126, 128) into the ears of ICR mice via I.D.

FIG. 6 shows a result of analyzing a protein expression efficiency by measuring an amount of R/L expressed 6 hours after administering lipid nanoparticles (LNP 412, 413, 414, 415, 416, 417, 418, 419, 420, 421), which is in a composition having a gallic acid derivative that includes Renilla Luciferase (R/L) mRNA, and positive control lipid nanoparticles (LNP 126, 128, 295, 334) into the ears of ICR mice via I.D.

FIG. 7 shows a result of measuring a concentration of MCP-1 from blood collected 6 hours after administering lipid nanoparticles (LNP 126, 128, 218, 219, 220, 221, 284, 285, 286, 287, 297, 298, 299, 300) that include mRNA encoding Renilla luciferase (R/L) into the ears of ICR mice via I.D.

FIG. 8 shows a result of measuring a concentration of MCP-1 from blood collected 6 hours after administering lipid nanoparticles (LNP 126, 222, 223, 288, 289, 301, 302, 316, 317, 318, 319, 320, 321, 343, 344, 347, 348) that include mRNA encoding Renilla luciferase (R/L) into the ears of ICR mice via I.D.

FIG. 9 shows a result of measuring a concentration of MCP-1 from blood collected 6 hours after administering lipid nanoparticles (LNP 126, 339, 340, 341, 342) that include mRNA encoding Renilla luciferase (R/L) into the ears of ICR mice via I.D.

FIG. 10 shows a result of measuring a concentration of MCP-1 from blood collected 6 hours after administering lipid nanoparticles (LNP 126, 128, 349, 350, 351, 352, 353, 354, 355, 356, 357, 358, 359, 360, 361, 362, 363, 364) that include mRNA encoding Renilla luciferase (R/L) into the ears of ICR mice via I.D.

FIG. 11 shows a result of measuring a concentration of MCP-1 from blood collected 6 hours after administering lipid nanoparticles (LNP 126, 128, 295, 334, 412, 413, 414, 415, 416, 417, 418, 419, 420, 421) that include mRNA encoding Renilla luciferase (R/L) into the ears of ICR mice via I.D.

### DETAILED DESCRIPTION

Hereinafter, the present disclosure will be described in detail.

The present inventor completed the present disclosure by preparing lipid nanoparticles using gallic acid-based lipid, one of the bio-friendly antioxidants, in an attempt to address issues of conventional lipid nanoparticles, and thus identifying the potential as a therapeutic agent capable of appropriately delivering nucleic acid drugs while reducing side effects caused by immune responses from the prepared lipid nanoparticles.

The present disclosure provides a lipid nanoparticle composition including a gallic acid derivative.

More specifically, the lipid nanoparticle composition according to the present disclosure may include a compound represented by the following Chemical Formula 1, a derivative thereof, or a pharmaceutically acceptable salt thereof as an active ingredient:

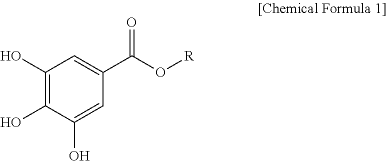

wherein, in the Chemical Formula 1, R may be a linear, saturated or unsaturated hydrocarbon with 3 to 22 carbon atoms.

Preferably, the compound may include any one or more selected from the group consisting of compounds represented by the following Chemical Formulas 2 to 6:

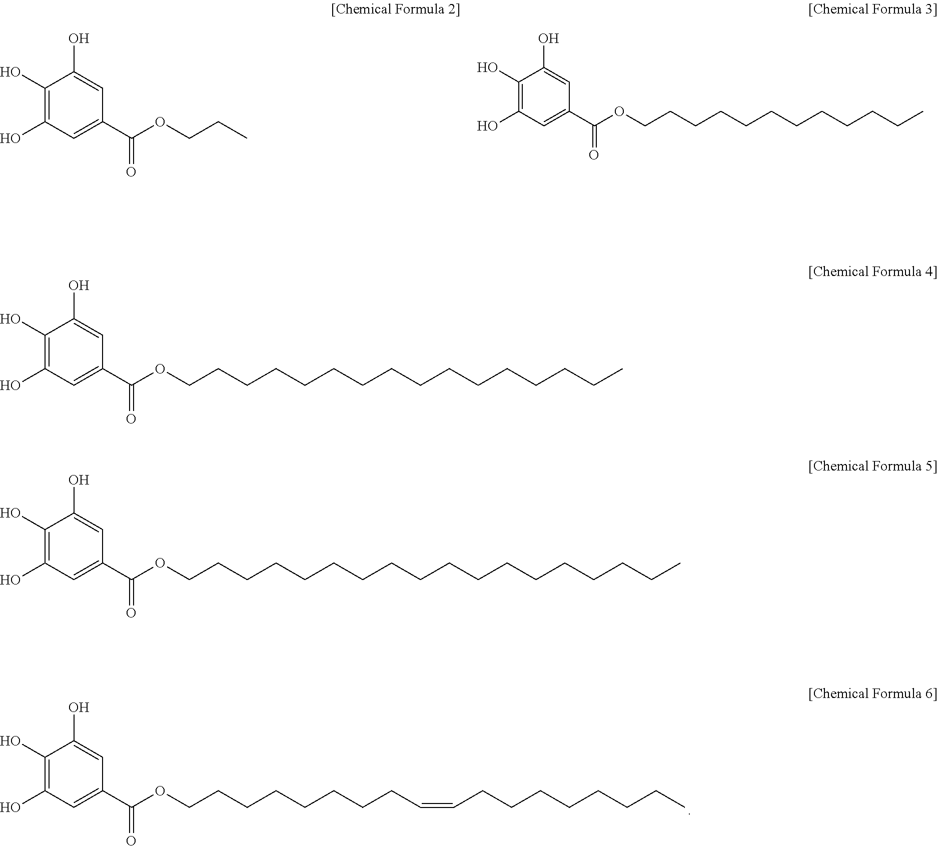 [SMILES: CCCOC(=O)c1cc(O)c(O)c(O)c1 ; CCCCCCCCCCCCOC(=O)c1cc(O)c(O)c(O)c1 ; CCCCCCCCCCCCCCCCOC(=O)c1cc(O)c(O)c(O)c1 ; CCCCCCCCCCCCCCCCCCOC(=O)c1cc(O)c(O)c(O)c1 ; CCCCCCCC/C=C\CCCCCCCCOC(=O)c1cc(O)c(O)c(O)c1]

In other words, the compound may include propyl gallate, dodecyl gallate, hexadecyl gallate, octadecyl gallate, cis-9-octadecenyl gallate, derivatives thereof, or pharmaceutically acceptable salts thereof.

The compound may be utilized as a helper lipid or structure-maintaining lipid of lipid nanoparticles.

The lipid nanoparticle composition according to the present disclosure may further include any one or more selected from the group consisting of an ionizable lipid, a helper lipid, a structure-maintaining lipid, a polyethylene glycol (PEG)-modified lipid, and an additive, in addition to the compound, a derivative thereof, or a pharmaceutically acceptable salt thereof.

The present disclosure provides a lipid nanoparticle composition including a compound represented by the following Chemical Formula 1, a derivative thereof, or a pharmaceutically acceptable salt thereof; an ionizable lipid; a helper lipid; and a polyethylene glycol (PEG)-modified lipid:

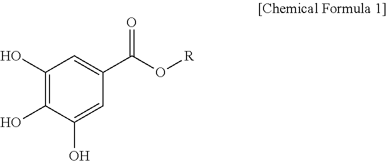

wherein, in the Chemical Formula 1, R may be selected from linear, saturated or unsaturated hydrocarbons with 3 to 22 carbon atoms, and the corresponding features may be substituted in the above-described part.

In the lipid nanoparticle composition according to the present disclosure, the ionizable lipid may be any one or more selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraene-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinoleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-diene-1-yloxy]propane-1-amine (octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (octyl-CLinDMA (2R)), (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (octyl-CLinDMA (2S)), 9-heptadecanyl 8-{(2-hydroxyethyl)[6-oxo-6-(undecyloxy)hexyl]amino}octanoate (SM-102), [(4-hydroxybutyl)azanediyl]di(hexane-6,1-diyl) bis(2-hexyldecanoate) (ALC-0315), and a pantothenic acid derivative, and preferably, it may be selected from SM-102 or a pantothenic acid derivative, but is not limited thereto.

The pantothenic acid derivative may be a compound selected from a compound represented by the following Chemical Formula 7, a stereoisomer thereof, a racemate thereof, or a pharmaceutically acceptable salt thereof:

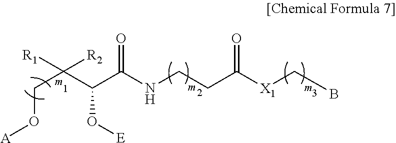

wherein, in the Chemical Formula 7, R₁ and R₂ are the same or different and each independently hydrogen or saturated hydrocarbons with 1 to 2 carbon atoms, X₁ is O, NH, or S, m₁, m₂, and m₃ are the same or different and each independently integers of 1 to 3, and A is hydrogen or a compound represented by the following Chemical Formula 7-1,

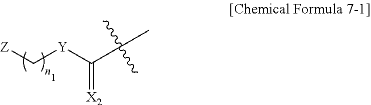

wherein, in the Chemical Formula 7-1, Z is NR¹R₂; a heterocyclic or aromatic compound with 3 to 8 carbon atoms; or a linear or branched, saturated or unsaturated hydrocarbon with 6 to 22 carbon atoms, wherein the R¹ and R² are the same or different and each independently linear or branched, saturated or unsaturated hydrocarbons with 1 to 6 carbon atoms; the heterocyclic or aromatic ring is substituted or unsubstituted by (C1˜C4)alkyl or di(C1˜C4)alkylamino(C1˜2)alkyl, the hydrocarbon includes or does not include ester, ether, amide, carbamate, carbonate, or disulfide bonds, X₂ is O or S, Y is CH₂, NH, or O, and n₁ is an integer from 0 to 3,
B is a compound represented by the following Chemical Formula 7-2 or NR^{1′}R^{2′}, and the R^{1′} and R^{2′} are the same or different and each independently linear or branched, saturated or unsaturated hydrocarbons with 1 to 6 carbon atoms, wherein the hydrocarbon includes or does not include ester, ether, amide, carbamate, carbonate, or disulfide bonds, and R^{1′} and R^{2′} are connected to each other to form a heterocyclic or aromatic ring with 3 to 8 carbon atoms,

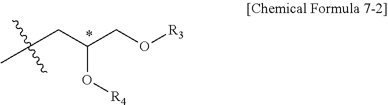

wherein, in the Chemical Formula 7-2, R₃ and R₄ are the same or different and each independently linear or branched, saturated or unsaturated hydrocarbons with 6 to 22 carbon atoms, wherein the hydrocarbon includes or does not include ester, ether, amide, carbamate, carbonate, or disulfide bonds,
E is hydrogen or a compound represented by the following Chemical Formula 7-3,

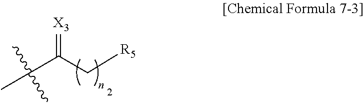

wherein, in the Chemical Formula 7-3, X₃ is O or S, n₂ is an integer of 0 to 3, and R₅ is a linear or branched, saturated or unsaturated hydrocarbon with 6 to 22 carbon atoms, wherein the hydrocarbon includes or does not include ester, ether, amide, carbamate, carbonate, or disulfide bonds, and may be substituted or unsubstituted by thiolane or dithiolane.

Specifically, the pantothenic acid derivative may include any one or more selected from the group consisting of compounds represented by the following Chemical formulas 8 to 13:

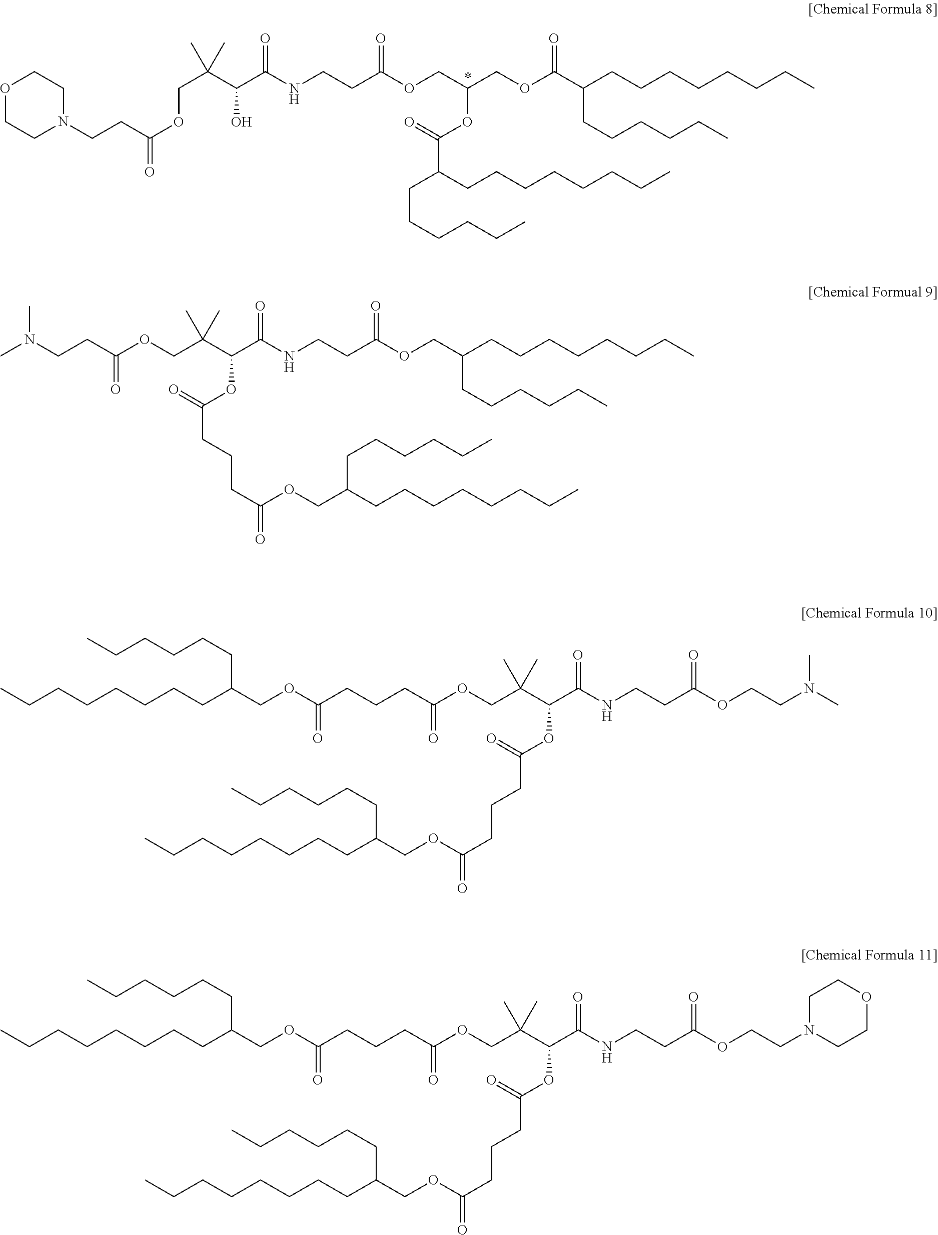

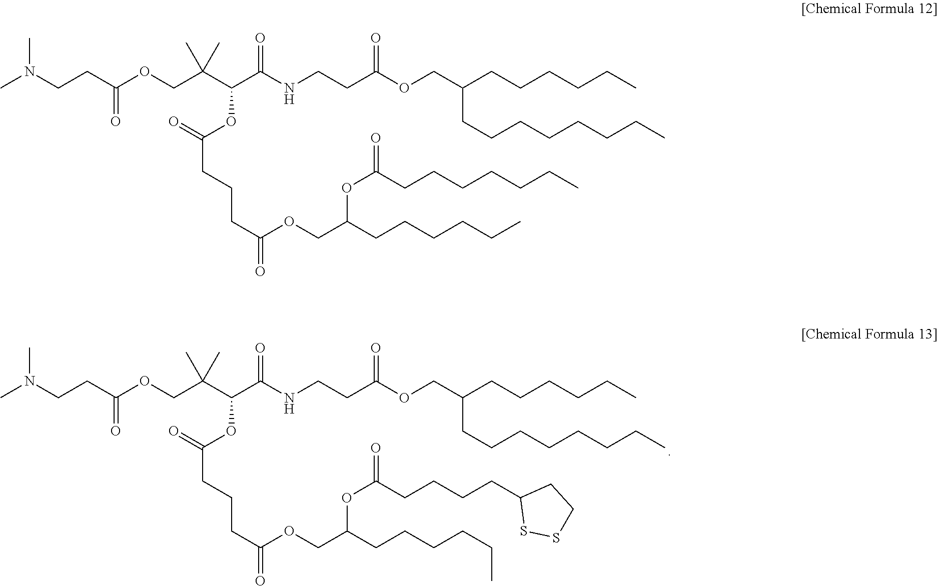

In the lipid nanoparticle composition according to the present disclosure, the helper lipid may be any one or more selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinoleoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinoleoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl-phosphatidyl-ethanolamine (DPPE), 1,2-dimyristoyl-sn-glycero-3-phosphoethanolamine (DMPE), 1-stearoyl-2-oleoyl-phosphatidylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and trehalose derivatives, and preferably, it may be selected from DOPE, DSPC, or trehalose derivatives, but is not limited thereto.

The trehalose derivative may include a compound represented by the following Chemical Formula 14:

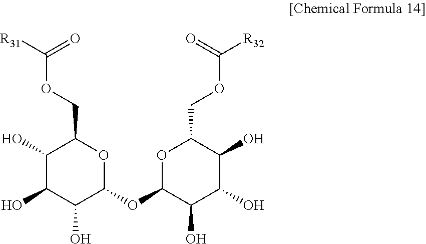

wherein, in the Chemical Formula 14, R₃₁ and R₃₂ may be the same or different and each independently selected from linear or branched, saturated or unsaturated hydrocarbons with 6 to 22 carbon atoms.

Specifically, the compound represented by Chemical Formula 14 may be a compound represented by the following Chemical Formula 15:

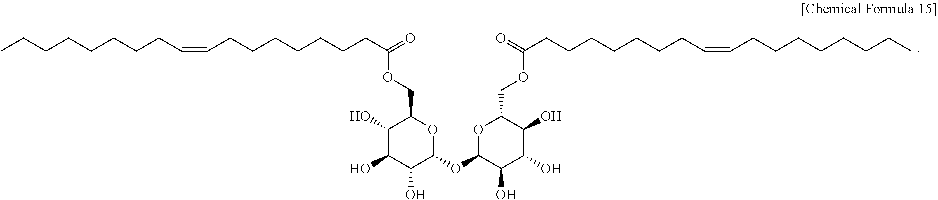 [SMILES: CCCCCCCC/C=C\CCCCCCCC(=O)OC[C@H]1O[C@H](O[C@H]2O[C@H](COC(=O)CCCCCCC/C=C\CCCCCCCC)[C@@H](O)[C@H](O)[C@H]2O)[C@H](O)[C@@H](O)[C@@H]1O]

In the lipid nanoparticle composition according to the present disclosure, the polyethylene glycol (PEG)-modified lipid may be selected from the group consisting of PEG-modified phosphatidylethanolamine, PEG-modified phosphatidic acid, PEG-modified ceramide, PEG-modified dialkylamine, PEG-modified diacylglycerol, PEG-modified dialkyl glycerol, and a mixture thereof, preferably it may be myristoyl diglyceride (DMG)-PEG, but is not limited thereto.

Preferably, the lipid nanoparticle composition according to the present disclosure may include 10 to 50 mol % of the compound, a derivative thereof, or a salt thereof, 20 to 60 mol % of the ionizable lipid, 5 to 20 mol % of the helper lipid, and 1 to 5 mol % of the PEG-modified lipid.

The composition may further include a structure-maintaining lipid, preferably from 1 to 40 mol %, but is not limited thereto.

The structure-maintaining lipid may be any one or more selected from the group consisting of cholesterol, bile acid derivatives including butyl lithocholate, cholanic acid derivatives, lithocholic acid derivatives, flavonoids, vitamin A and derivatives thereof, vitamin E, vitamin K, coenzyme Q10, and beta-carotene.

The lithocholic acid derivative may be a compound represented by the following Chemical Formula 16:

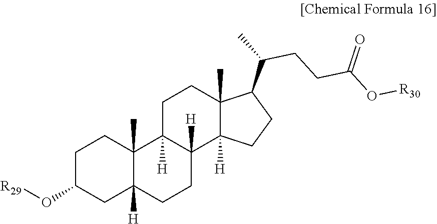

wherein, in the Chemical Formula 16, R₂₉ and R₃₀ may be the same or different and each independently linear or branched, saturated or unsaturated hydrocarbons with 1 to 22 carbon atoms, and at least any one or more of the R₂₉ and R₃₀ may be saturated or unsaturated hydrocarbons with 2 to 20 carbon atoms.

Specifically, the compound represented by Chemical Formula 16 may be a compound represented by the following Chemical Formula 17, where R₂₉ is hydrogen, and R₃₀ has an alkyl group with 4 carbon atoms:

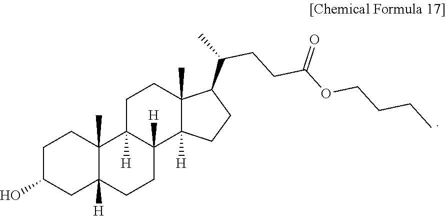

The present disclosure provides a composition for drug delivery, including the lipid nanoparticle composition; and a therapeutic or prophylactic agent.

The therapeutic or prophylactic agent may be a vaccine or compound capable of inducing an immune response.

Specifically, the therapeutic or prophylactic agent may be selected from the group consisting of small interfering RNA (siRNA), asymmetric interfering RNA (aiRNA), microRNA (miRNA), dicer-substrate RNA (dsRNA), small hairpin RNA (shRNA), messenger RNA (mRNA), and a mixture thereof.

The encapsulation efficiency of the therapeutic and/or prophylactic agent may independently be at least 50% or higher.

A wt/wt ratio of components of the lipid nanoparticle composition to the therapeutic or prophylactic agent may be from about 10:1 to about 60:1.

The N:P ratio of the therapeutic or prophylactic agent may be from about 2:1 to about 30:1. The N/P ratio is a value obtained by dividing the number of ionizable nitrogens in the ionizable lipid by the number of phosphate groups in nucleic acid molecules.

The present disclosure provides an immunoenhancing composition including the lipid nanoparticle composition.

Corresponding features may be substituted in the above-described part.

In addition, the present disclosure provides a method of delivering therapeutic and/or prophylactic agents to mammalian cells by the composition for drug delivery.

The method of delivering the therapeutic and/or prophylactic agent to the mammalian cell includes administering the lipid nanoparticle composition to a subject, wherein the administering may be conducted by bringing the cell in contact with the nanoparticle composition to allow the therapeutic and/or prophylactic agent to be delivered to the cell.

The mammalian cell is from mammals.

The mammal may be a human.

The composition for drug delivery may be administered intravenously, intramuscularly, intradermally, subcutaneously, intranasally, or by inhalation. About 0.01 mg/kg to about 10 mg/kg doses of the therapeutic and/or prophylactic agent may be administered to mammals.

Alternatively, the present disclosure may provide a drug delivery method that includes administering the composition for drug delivery into cells of animals other than humans.

Corresponding features may be substituted in the above-described part.

In addition, the present disclosure provides a method of producing a polypeptide of interest in mammalian cells.

The method of producing the polypeptide of interest in the mammalian cell includes brining the cell in contact with a composition for drug delivery including a lipid nanoparticle composition and a therapeutic and/or prophylactic agent, wherein the therapeutic and/or prophylactic agent is an mRNA, and the mRNA encodes the polypeptide of interest, such that the mRNA may be translated in cells to produce the polypeptide of interest.

Corresponding features may be substituted in the above-described part.

Hereinafter, to help the understanding of the present disclosure, example embodiments will be described in detail. However, the following example embodiments are merely illustrative of the content of the present disclosure, and the scope of the present disclosure is not limited to the following example embodiments. The example embodiments of the present disclosure are provided to more completely explain the present disclosure to those of ordinary skill in the art.

### <Preparation Example 1> Preparation of Gallic Acid Derivatives

Gallic acid derivative compounds represented by the following Chemical Formulas 2 to 6 were purchased and prepared from Tokyo Chemical Industry Co., Ltd.

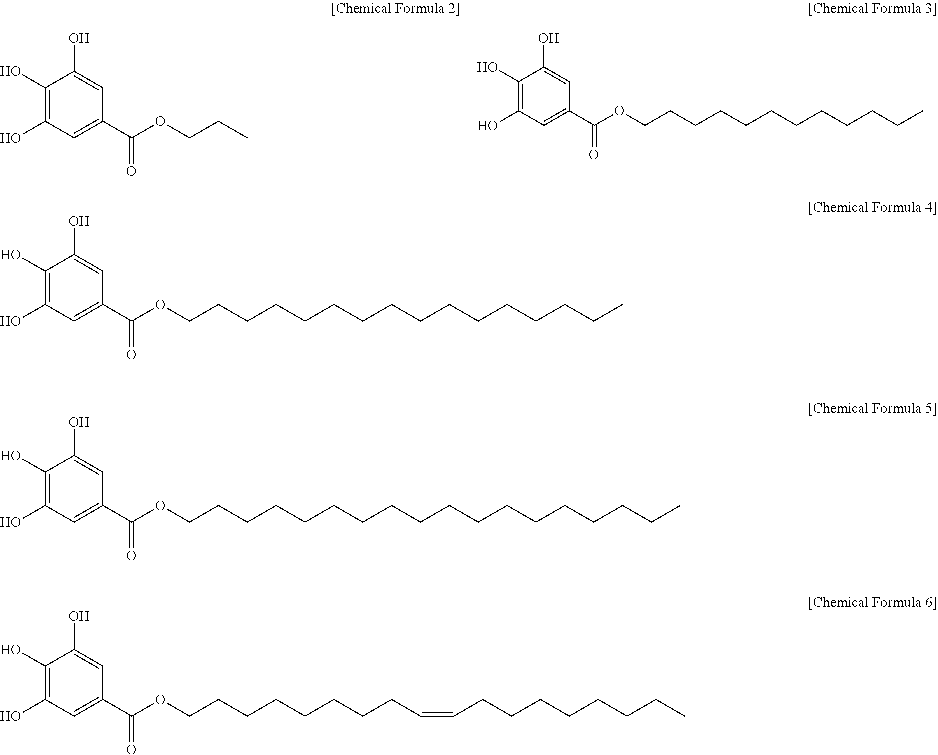 [SMILES: CCCOC(=O)c1cc(O)c(O)c(O)c1 ; CCCCCCCCCCCCOC(=O)c1cc(O)c(O)c(O)c1 ; CCCCCCCCCCCCCCCCOC(=O)c1cc(O)c(O)c(O)c1 ; CCCCCCCCCCCCCCCCCCOC(=O)c1cc(O)c(O)c(O)c1 ; CCCCCCCC/C=C\CCCCCCCCOC(=O)c1cc(O)c(O)c(O)c1]

### <Preparation Example 2> Preparation of Ionizable Lipid

9-Heptadecanyl 8-{(2-hydroxyethyl)[6-oxo-6-(undecyloxy)hexyl]amino}octanoate (SM-102) was purchased from Hanmi Fine Chemical Co., Ltd.

A pantothenic acid derivative was prepared by synthesis according to the following method.

### [Scheme 1] Synthesis of Compound 8

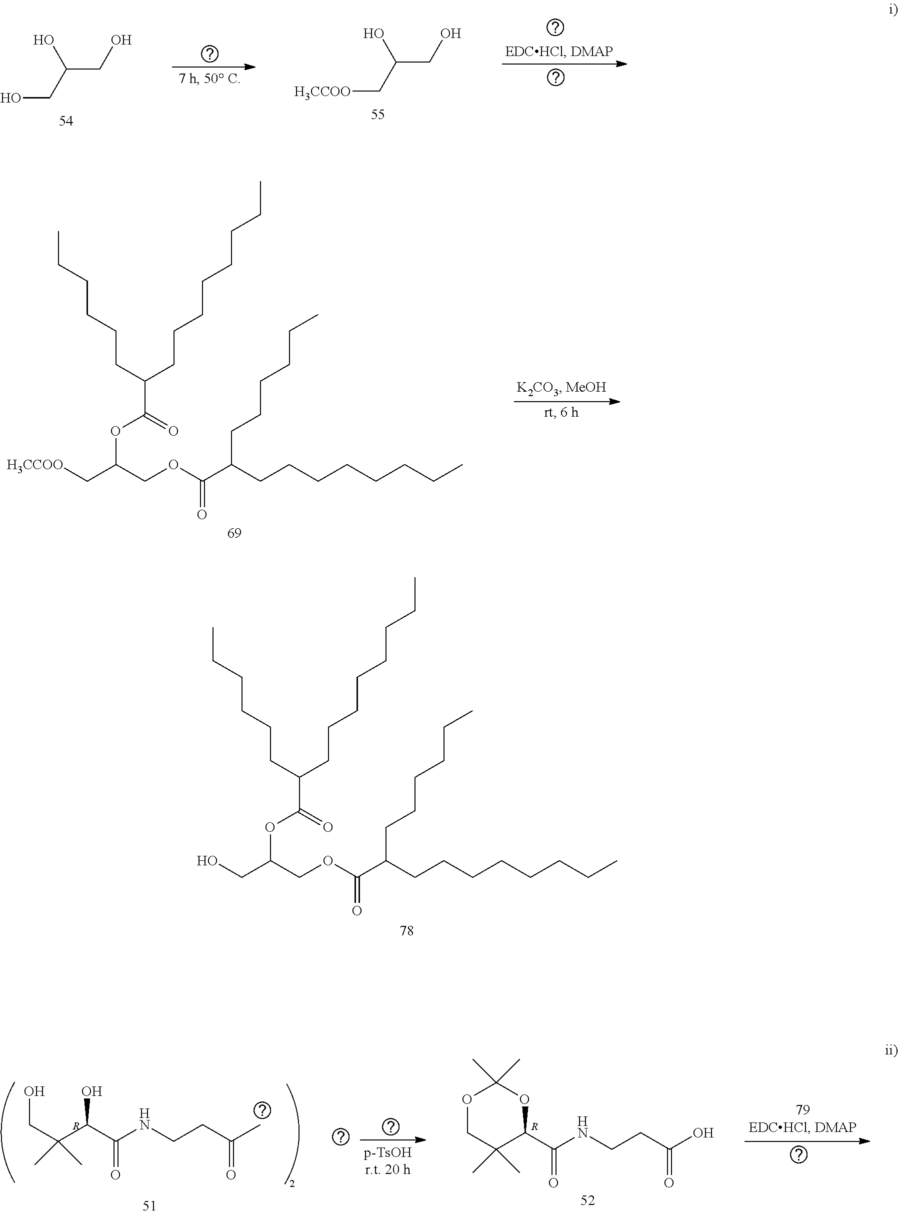

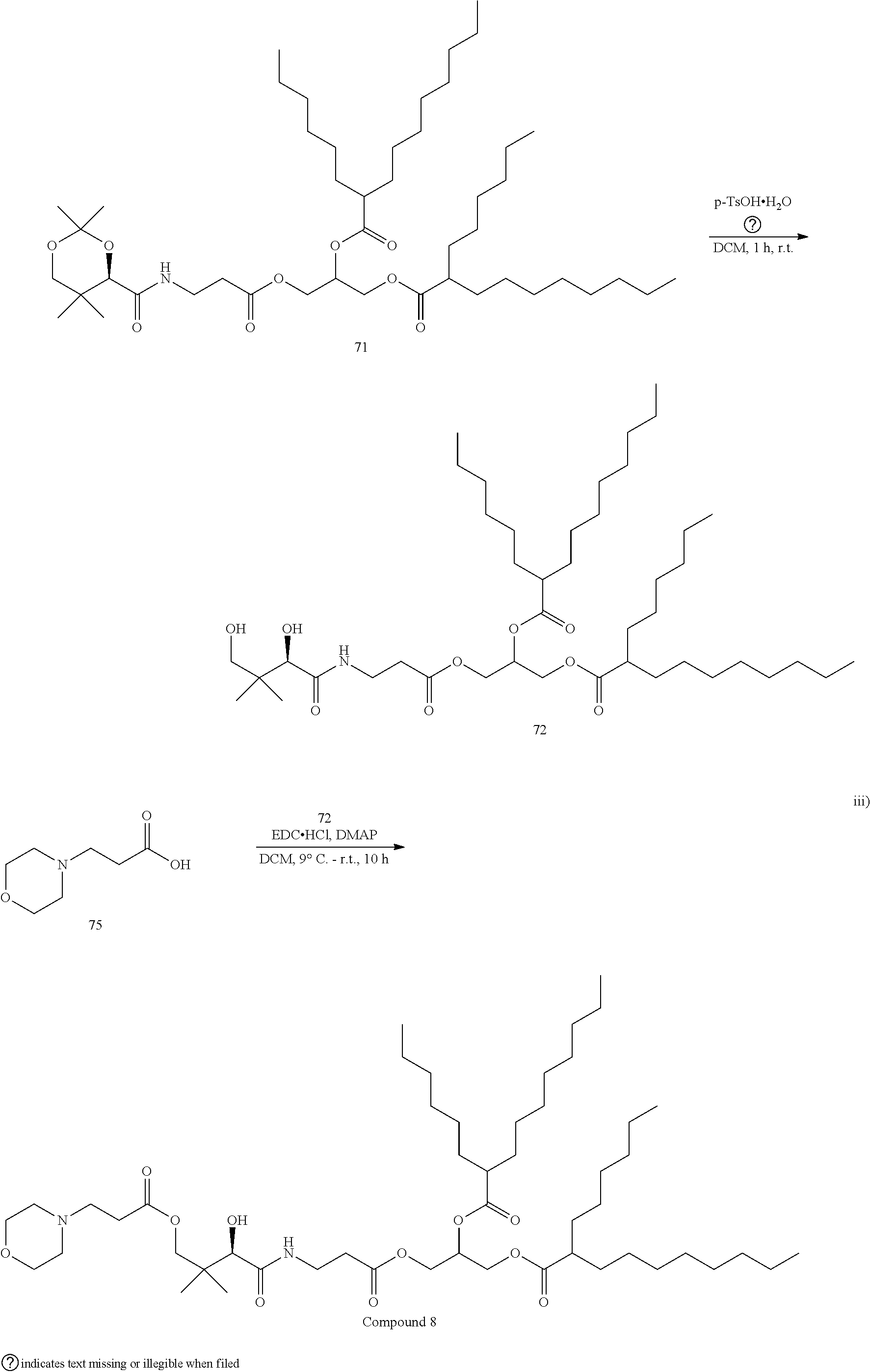 [SMILES: CCCCCCCCC(CCCCCC)C(=O)OCC(COC(=O)CCNC(=O)[C@@H]1OC(C)(C)OCC1(C)C)OC(=O)C(CCCCCC)CCCCCCCC ; CCCCCCCCC(CCCCCC)C(=O)OCC(COC(=O)CCNC(=O)[C@H](O)C(C)(C)CO)OC(=O)C(CCCCCC)CCCCCCCC ; O=C(O)CCN1CCOCC1 ; CCCCCCCCC(CCCCCC)C(=O)OCC(COC(=O)CCNC(=O)[C@H](O)C(C)(C)COC(=O)CCN1CCOCC1)OC(=O)C(CCCCCC)CCCCCCCC]

### i) Synthesis of Compound 70

### 2,3-Dihydroxypropyl acetate (55)

Propane-1,2,3-triol (Compound 54; 300 mg, 1 equiv.) and ethanoic anhydride (432 mg, 1.3 equiv.) were dissolved in an anhydrous acetonitrile (20 mL) solvent, then tetra-n-butylammonium acetate (TBAAc; 687 mg, 0.7 equiv.) was added, and the reaction mixture was stirred at 50° C. for 7 hours. After the termination of the reaction, the reaction mixture was loaded by a solid deposition method and purified by flash tube chromatography (SiO₂, n-hexane/EtOAc 5:5) to obtain Compound 55 (297 mg, 68%) in colorless oil.

¹H NMR (CDCl₃, 400 MHz): δ 2.11 (s, 3H), 2.8 (br s, 1H), 3.14 (br s, 1H), 3.59 (dd, 1H, J=6, 11.6 Hz), 3.69 (dd, 1H, J=3.6, 11.6 Hz), 3.93 (pnt, 1H, J=4.8 Hz), 4.22-4.12 (m, 2H); ¹³C NMR (CDCl₃, 100 MHz): δ 20.83, 63.31, 65.29, 70.12, 171.56.

### 3-Acetoxypropane-1,2-diyl bis(2-hexyldecanoate) (69)

2-Hexyldecanoic acid (956 mg, 2.5 equiv.) was put into a reaction vessel and then dissolved in DCM (40 mL), and EDCI·HCl (580 mg, 2.5 equiv.) and DMAP (55 mg, 0.3 equiv.) were added and stirred vigorously in the presence of argon gas at 5° C. for 10 minutes. Afterwards, a solution in which Compound 55 (200 mg, 1 equiv.) is dissolved in DCM (20 mL) was added dropwise to the reaction mixture at 5° C. and stirred at room temperature for 24 hours. After confirming that the reaction is terminated via TLC (SiO₂, EtOAc/hexane 4:6), DCM (50 mL) was added additionally, and the mixture was transferred to the separatory funnel, followed by washing with saturated NaHCO₃ solution (2×50 mL) and saturated NaCl solution (30 mL). A filtrate filtered by drying a separated organic layer with anhydrous MgSO₄ was distilled under reduced pressure to remove the solvent, and the mixture obtained was purified by flash column chromatography (SiO₂, EtOAc/hexane 1:9) to obtain Compound 69 (801 mg, 88%) in clear liquid.

¹H NMR (CDCl₃, 400 MHz): δ 0.82 (t, J=5.2 Hz, 12H), 1.18-1.25 (m, 42H), 1.36-1.41 (m, 5H), 1.49-1.55 (m, 5H), 2.11 (s, 3H), 2.25-2.31 (m, 2H), 3.5 (dd, J=3.6, 3.9 Hz, 1H), 3.62 (dd, J=3.6, 3.9 Hz, 1H), 4.13 (dd, J=3.4, 4.3 Hz, 1H), 4.30 (dd, J=3.4, 4.3 Hz, 1H), 5.12-5.16 (m, 1H); ¹³C NMR (CDCl₃, 100 MHz): δ 14.04, 14.08, 20.83, 22.61, 22.66, 27.31, 27.34, 27.31, 27.42, 27.46, 29.20, 29.23, 29.28, 29.45, 29.53, 29.56, 31.68, 32.31, 32.34, 32.19, 42.32, 45.65, 45.74, 62.09, 70.17, 171.56, 175.46, 175.87.

### 3-Hydroxypropane-1,2-diyl bis(2-hexyldecanoate) (70)

After glycerol Compound 69 (700 mg, 1 equiv.) was put into a reaction vessel and then clogged with a rubber stopper, nitrogen gas was filled and left until the termination of the reaction. After that, CH₃OH (70 mL) was added through a cannula, and then K₂CO₃ (316 mg, 2 equiv.) was added subsequently, followed by stirring at room temperature for 6 hours. The reaction mixture was distilled under reduced pressure to remove the solvent, then DCM (50 mL) and distilled water (50 mL) were added, and the mixture was transferred to the separatory funnel. The water layer was extracted with DCM (2×20 mL), and organic layers were collected and washed with saturated aqueous NaCl solution (20 mL). A filtrate filtered by drying the separated organic layers with anhydrous MgSO₄ was distilled under reduced pressure to remove the solvent, and the mixture obtained was purified by flash column chromatography (SiO₂, EtOAc/hexane 2:8->3:6) to obtain Compound 70 (502 mg, 77%) in clear oil.

¹H NMR (CDCl₃, 400 MHz): δ 0.81 (t, J=5.1 Hz, 12H), 1.18-1.24 (m, 42H), 1.36-1.41 (m, 5H), 1.49-1.57 (m, 5H), 2.25-2.31 (m, 2H), 3.56 (dd, J=3.6, 3.9 Hz, 1H), 3.62 (dd, J=3.6, 3.9 Hz, 1H), 4.12 (dd, J=3.4, 4.3 Hz, 1H), 4.30 (dd, J=3.4, 4.3 Hz, 1H), 5.12-5.16 (m, 1H); ¹³C NMR (CDCl₃, 100 MHz): δ 14.04, 14.08, 22.60, 22.66, 27.30, 27.34, 27.37, 27.41, 27.45, 29.19, 29.23, 29.28, 29.44, 29.53, 29.58, 31.68, 32.30, 32.33, 32.18, 42.32, 45.64, 45.73, 62.08, 70.16, 175.46, 175.86.

### ii) Synthesis of Compound 72

### (R)-3-(2,2,5,5-Tetramethyl-1,3-dioxane-4-carboxamido)propanoic acid (52)

While stirring D-pantothenic acid hemicalcium salt (Compound 51; 5 g, 1 equiv.) and 2,2-dimethoxypropane (DMP; 60 mL) in a reaction vessel, p-toluenesulfonic acid (PTSA; 3.97 g, 1.1 equiv.) was added at once. The liquid reaction mixture was stirred at room temperature for 20 hours, and the resulting white solids were filtered by washing with acetone. The yellow solid obtained by distilling the filtrate under reduced pressure was washed with warm n-hexane to obtain Compound 52 (5.45 g, 100%) in while solid.

¹H NMR (CDCl₃, 400 MHz): δ 0.98 (s, 3H), 1.04 (s, 3H), 1.43 (s, 3H), 1.46 (s, 3H), 2.62 (dt, J=6.0, 2.0, 2H), 3.29 (d, J=11.5, 1H), 3.45-3.53 (m, 1H), 3.56-3.64 (m, 1H), 3.70 (d, J=11.5, 1H), 4.11 (s, 1H), 7.05 (app br s, 1H); ¹³C NMR (CDCl₃, 100 MHz): δ 18.7, 18.8, 22.0, 29.4, 30.9, 33.0, 33.9, 34.1, 71.4, 77.1, 99.1, 170.2.

### 3-((3-((R)-2,2,5,5-Tetramethyl-1,3-dioxane-4-carboxamido)propanoyl)oxy)propane-1,2-diyl bis(2-hexyldecanoate) (71)

Pantothenic acid 52 (376 mg, 1.5 equiv.) protected with compound acetal was put into a reaction vessel and dissolved in DCM (30 mL), and then EDCI·HCl (225 mg, 1.5 equiv.) and DMAP (24 mg, 0.2 equiv.) were added and stirred vigorously in the presence of argon gas at 5° C. for 10 minutes. Thereafter, a solution, in which Compound 70 (550 mg, 1 equiv.) is dissolved in DCM (10 mL), was added dropwise in the reaction mixture at 5° C. and then stirred at room temperature for 11 hours. After confirming that the reaction was terminated via TLC (SiO₂, EtOAc/hexane 2:8), DCM (50 mL) was added additionally, and the mixture was transferred to the separatory funnel, followed by washing with saturated NaHCO₃ solution (2×50 mL) and saturated NaCl solution (30 mL). The filtrate filtered by drying a separated organic layer with anhydrous MgSO₄ was distilled under reduced pressure to remove the solvent, and then the mixture obtained was removed by column chromatography (SiO₂, EtOAc/hexane 3:7) to obtain Compound 71 (440 mg, 77%) in clear oil.

¹H NMR (CDCl₃, 400 MHz): δ 0.62 (t, J=5.1 Hz, 12H), 0.73 (s, 3H), 0.80 (s, 3H), 1.01-1.07 (m, 41H), 1.18-1.22 (m, 11H), 1.32-1.37 (m, 5H), 2.08-2.11 (m, 2H), 2.32 (t, J=4.1 Hz, 2H), 3.03 (d, J=6.1 Hz, 1H), 3.20-3.42 (m, 2H), 3.44 (d, J=6.1 Hz, 1H), 3.84-3.94 (m, 3H), 4.06-4.31 (m, 2H), 5.04-5.06 (m, 1H), 7.04 (s, 1H); ¹³C NMR (CDCl₃, 100 MHz): δ 14.03, 14.07, 18.66, 22.08, 22.58, 22.64, 27.26, 27.32, 27.37, 27.42, 29.18, 29.21, 29.27, 29.42, 29.54, 29.57, 31.65, 31.84, 32.21, 32.27, 32.95, 33.83, 33.85, 34.09, 32.12, 45.57, 45.63, 61.95, 62.72, 62.79, 68.64, 71.46, 99.00, 169.78, 171.55, 175.49, 175.87.

### 3-((3-((R)-2,4-Dihydroxy-3,3-dimethylbutanamido)propanoyl)oxy)propane-1,2-diyl bis(2-hexyldecanoate) (72)

Compound 71 (420 mg, 1 equiv.) and DTT (160 mg, 2 equiv.) were put into a reaction vessel and dissolved in DCM, and then p-toluenesulfonic acid (45 mg, 0.5 equiv.) was added at room temperature and stirred for 1 hour. Once the reaction was terminated, extraction was followed with EtOAc (30 mL×2), and organic layers were collected and washed with saturated NaCl solution. The filtrate filtered by drying the separated organic layers with anhydrous Na₂SO₄ was distilled under reduced pressure to remove the solvent, and then the mixture obtained was purified by flash column chromatography (SiO₂, EtOAc/hexane 3:7->6:4) to obtain Compound 72 (243 mg, 61%) in light yellow oil.

¹H NMR (CDCl₃, 400 MHz): δ 0.81 (t, J=5.1 Hz, 12H), 0.95 (s, 3H), 1.00 (s, 3H), 1.18-1.24 (m, 45H), 1.38 (m, 5H), 1.48-1.52 (m, 5H), 2.25-2.29 (m, 2H), 2.43-2.47 (m, 2H), 3.31-3.43 (m, 1H), 3.59-3.62 (m, 1H), 4.02-4.06 (m, 3H), 4.15-4.28 (m, 4H), 4.38-4.42 (m, 1H), 5.32-5.34 (m, 1H), 7.02-7.20 (m, 1H); ¹³C NMR (CDCl₃, 100 MHz): δ 14.03, 14.07, 19.91, 19.98, 20.84, 22.59, 22.65, 27.23, 27.28, 27.33, 27.38, 27.44, 29.11, 29.20, 29.27, 29.43, 29.56, 31.62, 31.65, 31.85, 32.21, 32.26, 32.28, 32.32, 32.43, 33.88, 34.41, 34.61, 38.29, 38.52, 45.62, 45.74, 61.95, 62.08, 62.80, 63.03, 68.70, 68.76, 73.27, 73.32, 74.70, 74.96, 169.66, 172.05, 173.54, 173.71.

### iii) Synthesis of Compound 8

### 3-((3-((R)-2-Hydroxy-3,3-dimethyl-4-((3-morpholinopropanoyl)oxy)butanamido) propanoyl)oxy)propane-1,2-diyl bis(2-hexyldecanoate) (Compound 8)

3-Morpholinopropanoic acid (Compound 75; 45.42 mg, 1.1 equiv.) was put into a reaction vessel and dissolved in DCM (20 mL), and then EDCI·HCl (75 mg, 1.5 equiv.) and DMAP (6.3 mg, 0.2 equiv.) were added and stirred vigorously in the presence of argon gas at room temperature for 20 minutes. Compound 72 (200 mg, 1 equiv.) was put into another reaction vessel and dissolved in DCM (10 mL) followed by cooling to 5° C., and then the liquid mixture of Compound 75 was added dropwise at 5° C. for 30 minutes and stirred at room temperature for 10 hours. After confirming that the reaction was terminated via TLC (SiO₂, EtOAc/MeOH 1:1 and 9:1, PMA stain), DCM (50 mL) was added additionally, and the mixture was transferred to the separatory funnel, followed by washing with saturated NaHCO₃ solution (2×50 mL) and saturated NaCl solution (20 mL). The filtrate filtered by drying a separated organic layer with anhydrous MgSO₄ was distilled under reduced pressure to remove the solvent, and then the mixture obtained was purified by flash column chromatography (SiO₂, DCM-MeOH 10:0->10:0.2, added with 28% aqueous NH₃ solution) to obtain Compound 8 (151 mg, 64%) in clear liquid.

¹H NMR (CDCl₃, 400 MHz): δ 0.79-0.82 (m, 15H), 1.09 (d, J=2.5 Hz, 3H), 1.18-1.24 (m, 43H), 1.34-1.39 (m, 4H), 1.48-1.51 (m, 4H), 2.24-2.28 (m, 2H), 2.41-2.52 (m, 10H), 3.48-3.63 (m, 7H), 3.85 (d, J=6 Hz, 1H), 4.00-4.04 (m, 2H), 4.23-4.37 (m, 4H), 5.23 (m, 1H), 7.19 (s, 1H); ¹³C NMR (CDCl₃, 100 MHz): δ 14.06, 14.10, 19.22, 19.27, 22.21, 22.61, 22.66, 27.34, 27.39, 24.44, 29.19, 29.24, 29.30, 29.45, 29.59, 31.68, 31.87, 32.22, 32.27, 33.93, 34.33, 34.37, 38.57, 38.65, 45.60, 45.66, 53.45, 54.62, 62.03, 62.80, 62.86, 66.22, 68.65, 71.04, 71.10, 74.42, 74.58, 171.64, 171.72, 172.17, 175.66, 175.84, 176.13, 176.08.

### [Scheme 2] Synthesis of Compound 9

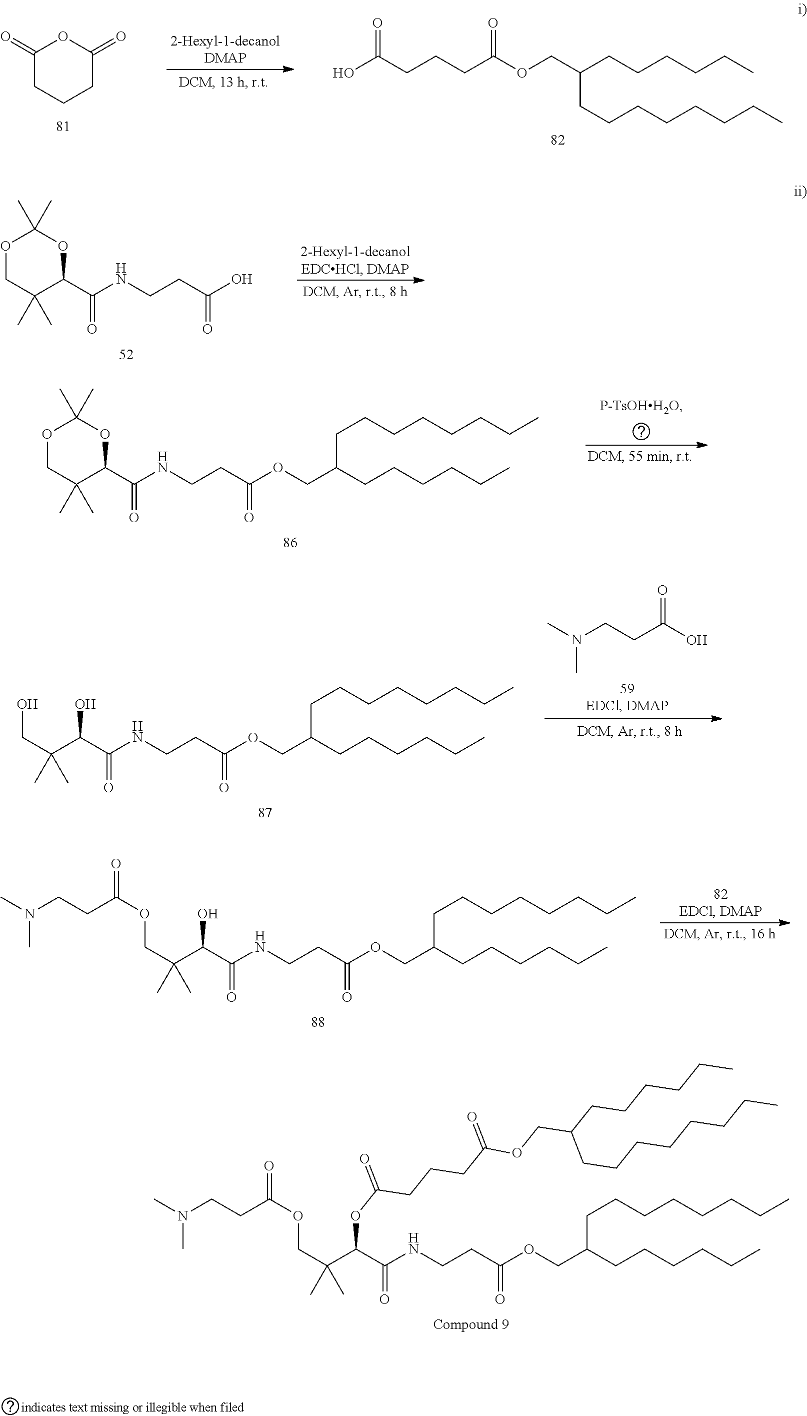

### i) Synthesis of Compound 82

### 5-((2-Hexyldecyl)oxy)-5-oxopentanoic acid (82)

2-Hexyl-1-decanoic acid (500 mg, 1 equiv.) was put into a reaction vessel and dissolved in DCM (50 mL), and then glutaric anhydride (Compound 81; 470 mg, 2 equiv.) and DMAP (630 mg, 2.5 equiv.) were added and stirred vigorously at room temperature for 13 hours. After confirming that the reaction was terminated via TLC (SiO₂, EtOAc/Hexane 1:1, PMA stain), 2N aqueous HCl solution (10 mL) was added to the liquid reaction mixture, followed by extraction with DCM (3×25 mL). All organic layers were collected and washed with 2N aqueous HCl solution (2×20 mL) to remove the residual bases, and then washing was followed with aqueous saturated NaCl solution (30 mL). The filtrate filtered by drying the separated organic layer with anhydrous Na₂SO₄ was distilled under reduced pressure to remove the solvent, and then the mixture was purified by flash column chromatography (SiO₂, EtOAc/hexane 1:9->2:8) to obtain Compound 82 (603 mg, 82%) in clear liquid.

¹H NMR (CDCl₃, 400 MHz): δ 0.81 (t, J=5.1 Hz, 6H), 1.19 (m, 25H), 1.54 (br s, 1H), 1.90 (qnt, J=5.2 Hz, 2H), 2.31-2.38 (m, 4H), 3.92 (d, J=7.2 Hz, 2H); ¹³C NMR (CDCl₃, 100 MHz): δ 14.07, 14.08, 19.86, 22.64, 22.67, 26.69, 29.30, 29.54, 29.60, 29.93, 31.25, 31.80, 31.89, 33.06, 33.25, 37.27, 67.39, 173.10, 179.25.

### ii) Synthesis of Compound 9

### 2-Hexyldecyl 3-((R)-2,2,5,5-tetramethyl-1,3-dioxane-4-carboxamido)propanoate (86)

Acetal-protected pantothenic acid Compound 52 (107 mg, 1.2 equiv.) was put into a reaction vessel and dissolved in DCM (30 mL), and then EDCI·HCl (119 mg, 1.5 equiv.) and DMAP (10 mg, 0.2 equiv.) were added and stirred vigorously in the presence of argon gas at 5° C. for 10 minutes. Afterwards, a solution in which 2-hexyldecan-1-ol (100 mg, 0.41 mmol) was dissolved in DCM (10 mL) was added dropwise at 5° C. and then stirred at room temperature for 8 hours. After confirming that the reaction was terminated via TLC (SiO₂, EtOAc/hexane 3:7, PMA stain), DCM (50 mL) was added additionally, and the mixture was transferred to the separatory funnel, followed by washing with saturated NaHCO₃ solution (2×50 mL) and saturated NaCl solution (20 mL). A filtrate filtered by drying a separated organic layer with anhydrous MgSO₄ was distilled under reduced pressure to remove the solvent, and the mixture obtained was purified by flash column chromatography (SiO₂, EtOAc/hexane 1:9->4:6) to obtain Compound 86 (177 mg, 89%) in clear oil.

¹H NMR (CDCl₃, 400 MHz): δ 0.89 (t, J=5.2 Hz, 3H), 0.97 (s, 3H), 1.05 (s, 3H), 1.27-1.32 (m, 25H), 1.43 (s, 3H), 1.47 (s, 3H), 1.62 (br s, 1H), 2.55-2.58 (m, 2H), 3.29 (d, J=9.2 Hz, 1H), 3.45-3.63 (m, 2H), 3.69 (d, J=8.1 Hz, 1H), 4.00 (d, J=9.8 Hz, 2H), 4.08 (s, 1H), 6.95-6.96 (m, 1H); ¹³C NMR (CDCl₃, 100 MHz): δ 14.07, 14.09, 18.68, 18.82, 22.09, 22.63, 22.66, 26.64, 26.67, 29.29, 29.46, 29.54, 29.58, 29.92, 31.20, 31.79, 31.87, 32.69, 34.14, 34.24, 37.27, 67.56, 71.49, 77.15, 98.99, 169.69, 172.30.

### 2-Hexyldecyl 3-((R)-2,4-dihydroxy-3,3-dimethylbutanamido) propanoate (87)

After dissolving Compound 86 (177 mg, 1 equiv.) and DTT (122 mg, 2 equiv.) in DCM, p-toluenesulfonic acid (64 mg, 0.5 equiv.) was added and stirred at room temperature for 55 minutes. Once the reaction was terminated, extraction was followed with EtOAc (2×30 mL), all organic layers were collected and washed with saturated NaCl solution, and then the filtrate filtered by drying the separated organic layers with anhydrous Na₂SO₄ was distilled under reduced pressure to remove the solvent. The remaining mixture was purified by flash column chromatography (SiO₂, EtOAc/hexane 1:1->7:3) to obtain Compound 87 (105 mg, 65%) in clear oil.

¹H NMR (CDCl₃, 400 MHz): δ 0.79-0.83 (m, 9H), 0.92 (s, 3H), 1.19-1.23 (m, 25H), 2.49 (t, J=6.2 Hz, 2H), 1.55 (br s, 1H), 3.38-3.54 (m, 4H), 3.91-3.94 (m, 3H), 7.22-7.25 (s, 1H); ¹³C NMR (CDCl₃, 100 MHz): δ 14.05, 14.03, 22.05, 22.64, 22.63, 26.64, 26.63, 29.29, 29.44, 29.55, 29.58, 29.91, 31.20, 31.79, 31.86, 32.69, 34.14, 34.24, 37.27, 67.56, 71.49, 77.15, 98.99, 169.69, 172.30.

### 2-Hexyldecyl 3-((R)-4-((3-(dimethylamino)propanoyl)oxy)-2-hydroxy-3,3-dimethylbutanamido)propanoate (88)

3-(Dimethylamino)propanoic acid (Compound 59; 29 mg, 1.1 equiv.) was put into a reaction vessel and dissolved in DCM (20 mL), and EDCI·HCl (69 mg, 1.6 equiv.) and DMAP (5 mg, 0.2 equiv.) were added and stirred vigorously in the presence of argon gas at room temperature for 20 minutes. Compound 87 (100 mg, 1 equiv.) was put into another reaction vessel, dissolved in DCM (10 mL), and cooled to 5° C., and then the liquid mixture of compound 59 was added dropwise at 5° C. for 30 minutes, followed by stirring at room temperature for 8 hours. After confirming that the reaction was terminated via TLC (SiO₂, EtOAc/MeOH 1:1, PMA stain), the reaction mixture was distilled under reduced pressure to remove the solvent, DCM (50 mL) was added additionally, and then the mixture was transferred to the separatory funnel, followed by washing with saturated NaHCO₃ solution (2×50 mL) and saturated NaCl solution (20 mL). A filtrate filtered by drying a separated organic layer with anhydrous MgSO₄ was distilled under reduced pressure to remove the solvent, and the mixture obtained was purified by flash column chromatography (SiO₂, DCM/MeOH 9:1/28% aqueous NH₃ solution) to obtain Compound 86 (177 mg, 89%) in clear oil.

¹H NMR (CDCl₃, 400 MHz): δ 0.79-0.82 (m, 9H), 1.12 (s, 3H), 1.19-1.31 (m, 25H), 1.53 (br. s, 1H), 2.16 (s, 6H), 2.38-2.66 (m, 6H), 3.38-3.53 (m, 3H), 3.81 (s, 1H), 3.91 (d, J=6.2 Hz, 2H), 4.25 (d, J=8.2 Hz, 1H), 7.40 (s, 1H); ¹³C NMR (CDCl₃, 100 MHz): δ 14.08, 14.10, 19.31, 22.49, 22.63, 22.66, 26.64, 26.68, 29.29, 29.55, 29.58, 29.93, 31.18, 31.80, 31.88, 33.33, 34.22, 34.56, 37.25, 38.48, 37.25, 38.48, 45.05, 55.75, 67.56, 71.49, 74.47, 172.17, 172.24, 172.62.

### (9R)-17-Hexyl-2,8,8-trimethyl-5,10,14-trioxo-6,15-dioxa-2,11-diazapentacosan-9-yl(2-hexyldecyl)glutarate (Compound 9)

Compound 82 (71 mg, 1.3 equiv.) was put into a reaction vessel and dissolved in DCM (30 mL), and EDCI·HCl (45 mg, 1.5 equiv.) and DMAP (19 mg, 0.2 equiv.) were added and stirred vigorously in the presence of argon gas at 5° C. for 10 minutes. Thereafter, a solution in which Compound 88 (84 mg, 1 equiv.) is dissolved in DCM (10 mL) was added dropwise into the reaction mixture at 5° C., followed by stirring at room temperature for 16 hours. After confirming that the reaction was terminated via TLC (SiO₂, EtOAc/hexane 5:5, PMA stain), DCM (50 mL) was added additionally, and the mixture was transferred to the separatory funnel, followed by washing with saturated NaHCO₃ solution (2×50 mL) and saturated NaCl solution (20 mL). A filtrate filtered by drying a separated organic layer with anhydrous MgSO₄ was distilled under reduced pressure to remove the solvent, the remaining mixture was purified by flash column chromatography (SiO₂, EtOAc/hexane 5:5->6:4) to obtain Compound 9 (120 mg, 88%) in clear oil.

¹H NMR (CDCl₃, 400 MHz): δ 0.79-0.82 (m, 12H), 0.98-0.99 (m, 6H), 1.11-1.36 (m, 47H), 1.53 (br. s, 2H), 1.90 (qnt, J=7.3 Hz, 2H), 2.21 (s, 6H), 2.31 (t, J=6.7 Hz, 2H), 2.41-2.65 (m, 8H), 3.27-3.32 (m, 1H), 3.46-3.53 (m, 1H), 3.74 (d, J=9.3 Hz, 1H), 3.38-3.94 (m, 5H), 4.79 (s, 1H), 7.38 (s, 1H); ¹³C NMR (CDCl₃, 100 MHz): δ 14.09, 20.05, 20.27, 22.04, 22.64, 22.66, 26.64, 26.69, 23.30, 29.56, 29.60, 29.94, 31.16, 31.21, 31.81, 31.88, 33.09, 33.24, 33.78, 35.04, 37.20, 37.24, 37.27, 24.21, 55.13, 67.33, 67.57, 69.59, 76.61, 76.73, 77.05, 77.25, 167.84, 171.69, 191.97, 172.43, 173.04.

### [Scheme 3] Synthesis of Compound 10

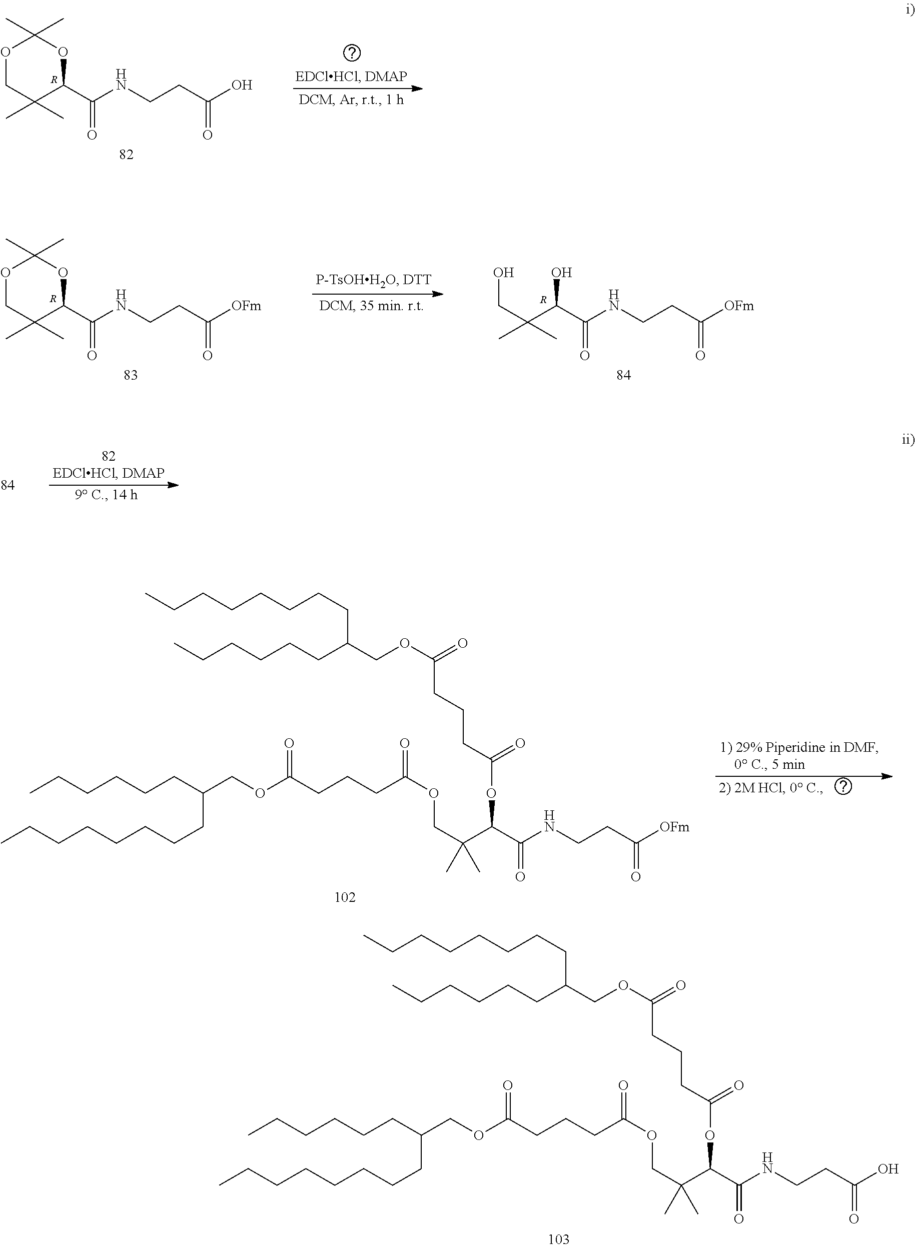

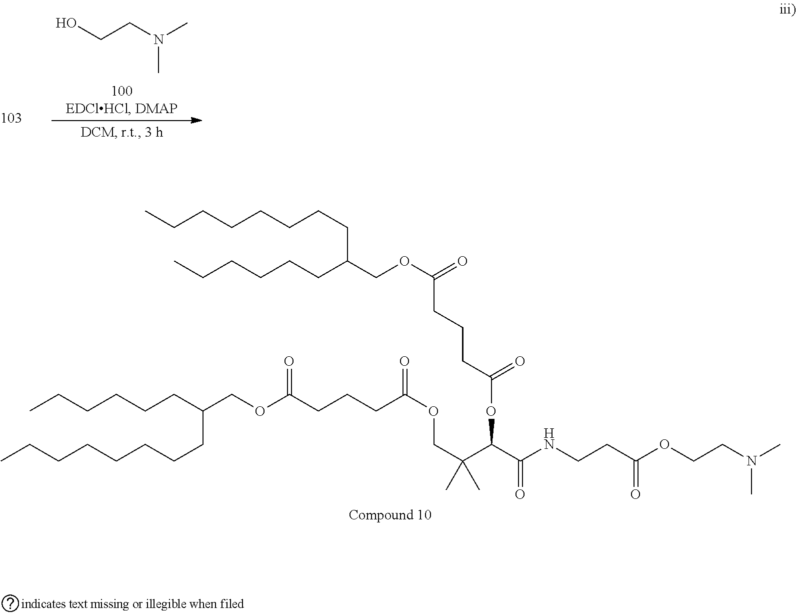

### i) Synthesis of Compound 94

### (9H-Fluoren-9-yl)methyl (R)-3-(2,2,5,5-tetramethyl-1,3-dioxane-4-carboxamido)propanoate (93)

Acetal-protected pantothenic acid compound 52 (476 mg, 1.2 equiv.) was put into a reaction vessel and dissolved in DCM (30 mL), and then EDCI·HCl (438 mg, 1.5 equiv.) and DMAP (38 mg, 0.2 equiv.) were added and stirred vigorously in the presence of argon gas at 5° C. for 10 minutes. Thereafter, a solution in which 9-fluorenemethanol (300 mg, 1 equiv.) is dissolved in DCM (10 mL) was added dropwise into the reaction mixture at 5° C., followed by stirring at room temperature for 1 hour. After confirming that the reaction was terminated via TLC (SiO₂, EtOAc/hexane 1.9, PMA stain), DCM (50 mL) was added additionally, and the mixture was transferred to the separatory funnel, followed by washing with saturated NaHCO₃ solution (2×50 mL) and saturated NaCl solution (20 mL) sequentially. A filtrate filtered by drying a separated organic layer with anhydrous MgSO₄ was distilled under reduced pressure to remove the solvent, and the mixture obtained was purified by flash column chromatography (SiO₂, EtOAc/hexane 1.9) to obtain Compound 93 (541 mg, 81%) in clear oil.

¹H NMR (CDCl₃, 400 MHz): δ 0.86 (s, 3H), 0.94 (s, 3H), 1.27 (s, 3H), 1.31 (s, 3H), 2.56 (t, J=8.2 Hz, 2H), 3.16 (d, J=5.1 Hz, 1H), 3.38-3.52 (m, 2H), 3.56 (d, J=5.5 Hz, 1H), 3.98 (s, 1H), 4.09 (t, J=8.3 Hz, 1H), 4.28 (d, J=7.1 Hz, 2H), 6.89 (s, 1H), 7.17 (t, J=5.1 Hz, 2H), 7.24 (t, J=5.5 Hz, 2H), 7.38 (d, J=5.0 Hz, 2H), 7.57 (d, J=4.8 Hz, 2H); ¹³C NMR (CDCl₃, 100 MHz): δ 14.23, 18.67, 18.89, 21.03, 22.14, 29.45, 32.79, 32.98, 34.21, 34.25, 46.72, 46.92, 60.36, 66.66, 71.45, 76.87, 77.19, 77.50, 99.02, 119.78, 120.09, 120.35, 124.69, 125.01, 127.16, 127.87, 141.29, 143.64, 169.82, 171.06.

### (9H-Fluoren-9-yl)methyl (R)-3-(2,4-dihydroxy-3,3-dimethylbutanamido)propanoate (94)

After dissolving Compound 93 (500 mg, 1 equiv.) and DTT (352 mg, 2 equiv.) in DCM, p-toluenesulfonic acid (98 mg, 0.5 equiv.) was added at room temperature and stirred for 15 minutes. Once the reaction was terminated, the liquid reaction mixture was extracted with EtOAc (30 mL×2) and then washed with saturated NaCl solution after collecting all organic layers. A filtrate filtered by drying the separated organic layers with anhydrous Na₂SO₄ was distilled under reduced pressure to remove the solvent, and the mixture obtained was purified by flash column chromatography (SiO₂, EtOAc/hexane 1:1->7:3) to obtain Compound 94 (277 mg, 61%) in clear oil.

¹H NMR (CDCl₃, 400 MHz): δ 0.90 (s, 3H), 0.97 (s, 3H), 2.63 (t, J=8.1 Hz, 2H), 3.47-3.58 (m, 4H), 4.00 (s, 1H), 4.20 (t, J=5.1 Hz, 1H), 4.41 (d, J=7.1 Hz, 2H), 7.27-7.30 (m, 3H), 7.33 (t, J=5.5 Hz, 2H), 7.41 (d, J=5.0 Hz, 2H), 7.67 (d, J=4.8 Hz, 2H); ¹³C NMR (CDCl₃, 100 MHz): δ 14.19, 20.34, 21.05, 21.31, 34.06, 34.63, 39.26, 46.71, 53.47, 60.47, 66.62, 71.15, 76.82, 77.14, 77.45, 77.48, 120.09, 124.95, 127.18, 127.89, 141.29, 143.59, 171.33, 172.17, 173.54.

### ii) Synthesis of Compound 103

### O,O′-(4-((3-((9H-Fluoren-9-yl)methoxy)-3-oxopropyl)amino)-2,2-dimethyl-4-oxobutane-1,3-diyl) bis(2-hexyldecyl) diglutarate (102)

Compound 82 (164 mg, 2.2 equiv.) was put into in a reaction vessel and dissolved in DCM (30 mL), and then EDCI·HCl (101 mg, 1.5 equiv.) and DMAP (7 mg, 0.2 equiv.) were added and stirred vigorously in the presence of argon gas at 5° C. for 10 minutes. Subsequently, a solution in which Compound 94 (250 mg, 1 equiv.) is dissolved in DCM (10 mL) was added into the reaction mixture dropwise at 5° C., followed by stirring at room temperature for 4 hours. After confirming that the reaction was terminated via TLC (SiO₂, EtOAc/hexane 1:9, PMA stain), DCM (100 mL) was added additionally, followed by washing with saturated NaHCO₃ solution (2×200 mL) and saturated NaCl solution (200 mL) sequentially. A filtrate filtered by drying a separated organic layer with anhydrous MgSO₄ was distilled under reduced pressure to remove the solvent, and the mixture obtained was purified by flash column chromatography (SiO₂, EtOAc/hexane 1:9) to obtain Compound 102 (300 mg, 81%)) in clear oil.

¹H NMR (CDCl₃, 400 MHz): δ 0.80 (t, J=8.0 Hz, 12H), 0.95 (d, J=9.2 Hz, 6H), 1.11-1.28 (m, 50H), 1.52 (s, 2H), 1.63 (s, 1H), 2.00 (qnt, J=5.5 Hz, 4H), 2.26-2.33 (m, 6H), 2.38 (t, J=5.7 Hz, 2H), 2.55 (t, J=5.9 Hz, 2H), 3.38-3.51 (m, 2H), 3.76-3.97 (m, 6H), 4.22 (t, J=5.0 Hz, 1H), 4.33 (d, J=9.1 Hz, 2H), 4.88 (s, 1H), 6.61 (m, 1H), 7.24 (t, J=7.1 Hz, 2H), 7.26 (t, J=8.1 Hz, 2H), 7.49 (d, J=8.3 Hz, 2H), 7.68 (d, J=8.2 Hz, 2H); ¹³C NMR (CDCl₃, 100 MHz): δ 14.08, 14.10, 20.00, 20.13, 20.90, 21.32, 22.64, 22.67, 26.65, 26.69, 29.30, 29.56, 29.60, 29.95, 31.21, 31.24, 31.81, 31.89, 33.05, 33.17, 33.24, 33.37, 33.66, 34.65, 37.29, 37.38, 46.70, 66.71, 67.35, 67.41, 69.23, 120.08, 124.98, 125.00, 127.17, 127.88, 141.30, 143.57, 143.65, 167.95, 171.62, 172.48, 172.62, 173.01, 173.09.

### 3-(2,4-bis((5-((2-Hexyldecyl)oxy)-5-oxopentanoyl)oxy)-3,3-dimethylbutanamido)propanoic acid (103)

After mixing and stirring piperidine and DMF in a volume ratio of 2:8 in a reaction vessel, the mixture was cooled to 0° C. to prepare 20% piperidine solution. Compound 102 (300 mg) was prepared by dissolving in DMF (20 mL) in another reaction vessel, and then the mixture was stirred for 5 minutes and cooled to 0° C. The 20% piperidine solution (40 mL) previously prepared was added under argon gas and stirred at 0° C. for 5 minutes. Once the reaction was terminated, 3M aqueous HCl solution was added into the reaction mixture, pH was adjusted to 7, EtOAc (200 mL) was added, and the mixture was transferred to the separatory funnel, followed by washing with distilled water (2×200 mL) and saturated NaCl solution (2×200 mL) sequentially. A filtrate filtered by drying a separated organic layer with anhydrous Na₂SO₄ was distilled under reduced pressure to remove the solvent, and the mixture obtained was purified by flash column chromatography (SiO₂, EtOAc/hexane 0:1->4:6, 1% AcOH added) to obtain Compound 103 (226 mg, 91%) in clear oil.

¹H NMR (CDCl₃, 400 MHz): δ 0.81 (t, J=8.1 Hz, 12H), 0.92 (d, J=9.0 Hz, 6H), 1.12-1.21 (m, 51H), 1.52 (s, 2H), 1.62 (s, 1H), 2.30 (m, 6H), 2.39 (t, J=5.8 Hz, 2H), 2.54 (t, J=5.3 Hz, 2H), 3.38-3.52 (m, 2H), 3.77-3.97 (m, 6H), 4.89 (s, 1H), 6.62 (m, 1H); ¹³C NMR (CDCl₃, 100 MHz): δ 14.09, 14.11, 20.02, 20.14, 20.91, 21.31, 22.66, 22.68, 26.61, 26.65, 29.31, 29.56, 29.61, 29.95, 31.22, 31.24, 31.81, 31.89, 33.05, 33.17, 33.24, 33.37, 33.66, 34.65, 37.29, 37.38, 46.70, 66.71, 67.35, 67.41, 69.23, 167.95, 171.62, 172.48, 172.62, 173.01, 173.09.

### iii) Synthesis of Compound 10

### O,O′-(4-((3-((2-(Dimethylamino)ethyl)amino)-3-oxopropyl)amino)-2,2-dimethyl-4-oxobutane-1,3-diyl) bis(2-hexyldecyl) diglutarate (Compound 10)

Compound 103 (100 mg, 1 equiv.) was put into a reaction vessel and dissolved in DCM (30 mL), and then EDCI·HCl (33 mg, 1.5 equiv.) and DMAP (3 mg, 0.2 equiv.) were added and stirred vigorously in the presence of argon gas at 5° C. for 10 minutes. Subsequently, a solution in which Compound 100 (11 mg, 1.1 equiv.) is dissolved in DCM (10 mL) was added to the reaction mixture dropwise at 5° C., followed by stirring at room temperature for 4 hours. After confirming that the reaction was terminated via TLC (SiO₂, EtOAc 100%, PMA stain), DCM (100 mL) was added additionally, followed by washing with saturated NaHCO₃ solution (2×200 mL) and saturated NaCl solution (200 mL) sequentially. A filtrate filtered by drying a separated organic layer with anhydrous MgSO₄ was distilled under reduced pressure and purified by column chromatography (SiO₂, EtOAc/MeOH 20:0->19:1) to obtain Compound 10 (97 mg, 90%) in clear oil.

¹H NMR (CDCl₃, 400 MHz): δ 0.82 (t, J=7.1 Hz, 12H), 0.97 (d, J=8.1 Hz, 6H), 1.11-1.32 (m, 50H), 1.54 (s, 2H), 1.88 (qnt, J=8.0 Hz, 4H), 2.22 (s, 6H), 2.31 (t, J=7.0 Hz, 6H), 2.39 (t, J=7.9 Hz, 2H), 2.43-2.51 (m, 4H), 3.38-3.51 (m, 2H), 3.37 (d, J=9.6 Hz, 2H), 3.91 (d, J=9.7 Hz, 4H), 3.96 (d, J=9.9 Hz, 2H), 4.10-4.14 (m, 2H), 4.87 (s, 1H), 6.77 (t, J=6.9 Hz, 1H); ¹³C NMR (CDCl₃, 100 MHz): δ 14.04, 19.99, 20.09, 20.79, 21.32, 22.60.22.62, 26.61, 26.66, 29.26, 29.52, 29.56, 29.91, 31.20, 31.77, 31.85, 33.02, 33.16, 33.19, 33.33, 33.75, 34.66, 37.26, 37.30, 45.57, 57.68, 62.16, 67.28, 67.34, 69.21, 167.80, 171.59, 172.44, 192.55, 172.95, 173.01.

### [Scheme 4] Synthesis of Compound 11

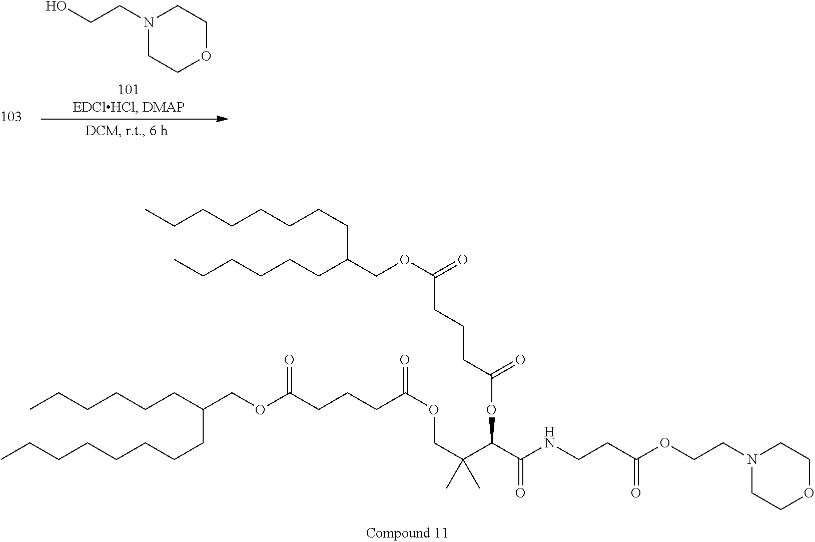 [SMILES: OCCN1CCOCC1 ; CCCCCCCCC(CCCCCC)COC(=O)CCCC(=O)OCC(C)(C)[C@@H](OC(=O)CCCC(=O)OCC(CCCCCC)CCCCCCCC)C(=O)NCCC(=O)OCCN1CCOCC1]

### O,O′-(2,2-Dimethyl-4-((3-((2-morpholinoethyl)amino)-3-oxopropyl)amino)-4-oxobutane-1,3-diyl) bis(2-hexyldecyl) diglutarate (Compound 11)

After dissolving Compound 103 (100 mg, 1.3 equiv.) in DCM (30 mL) in the reaction vessel, EDCI·HCl (33 mg, 1.5 equiv.) and DMAP (3 mg, 0.2 equiv.) were added and stirred vigorously in the presence of argon gas at 5° C. for 10 minutes. Subsequently, a solution in which Compound 101 (17 mg, 1.1 equiv.) is dissolved in DCM (10 mL) was added in the reaction mixture dropwise at 5° C. and stirred for 6 hours at room temperature. After confirming that the reaction was terminated via TLC (SiO₂, EtOAc 100%, PMA stain), DCM (100 mL) was added additionally, followed by washing with saturated NaHCO₃ solution (2×200 mL) and saturated NaCl solution (1×200 mL) sequentially. A mixture obtained by distilling under reduced pressure a filtrate filtered by drying a separated organic layer with anhydrous MgSO₄ was purified by flash column chromatography (SiO₂, EtOAc/hexane 8:3->10:0) to obtain Compound 11 (89 mg, 79%) in clear oil.

¹H NMR (CDCl₃, 400 MHz): δ 0.81 (t, J=7.6 Hz, 12H), 0.96 (d, J=8.7 Hz, 6H), 1.10-1.31 (m, 47H), 1.54 (s, 2H), 1.85-1.93 (m, 4H), 2.28-2.57 (m, 16H), 2.33-2.50 (m, 2H), 3.65 (m, 4H), 3.78 (d, 1H), 3.89-3.98 (m, 5H), 4.13-1.19 (m, 2H), 4.88 (s, 1H); ¹³C NMR (CDCl₃, 100 MHz): δ 14.06, 20.01, 20.12, 20.89, 21.31, 22.62, 22.64, 26.64, 26.68, 29.28, 29.54, 29.58, 29.93, 31.22, 31.79, 31.87, 33.06, 33.18, 33.23, 33.35, 33.66, 34.68, 37.29, 37.35, 53.76, 56.97, 61.62, 66.72, 67.34, 37.41, 69.21, 167.88, 171.62, 172.38, 172.59, 173.00, 173.05.

### [Scheme 5] Synthesis of Compound 12

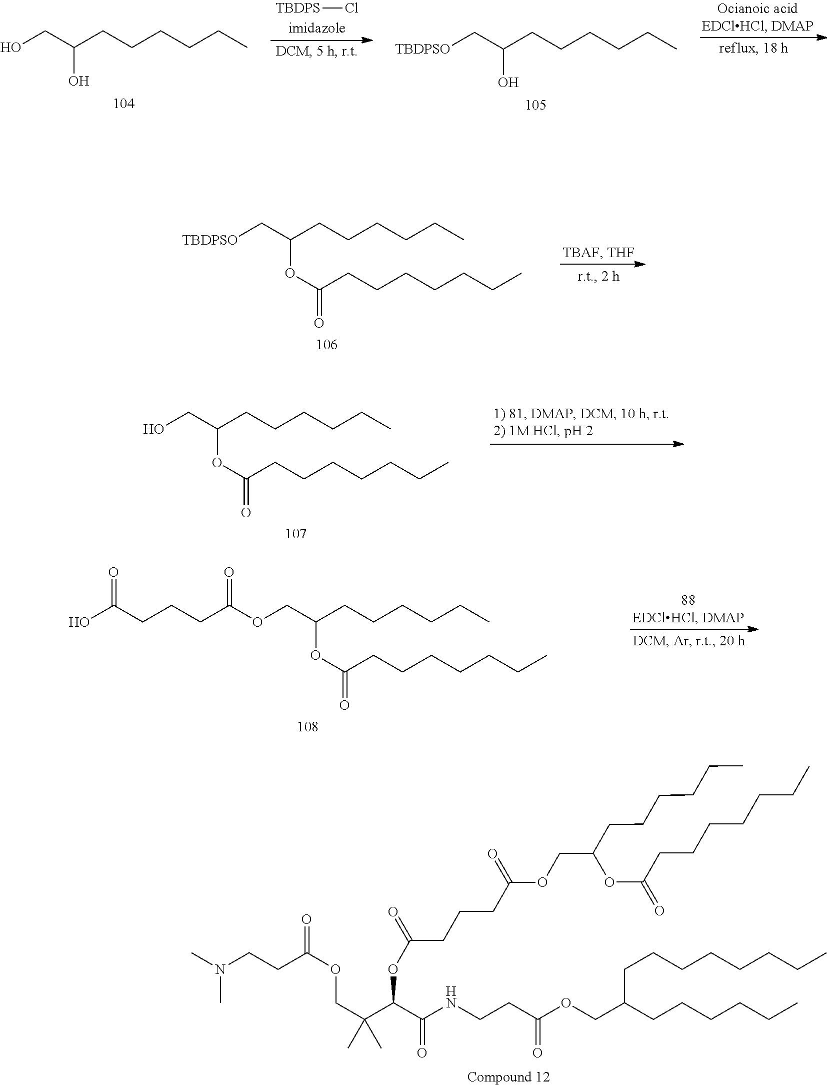

### 1-((tert-Butyldiphenylsilyl)oxy)octan-2-ol (105)

1,2-Octanediol (Compound 104; 500 mg, 1 equiv.) and imidazole (279.32, 1.2 equiv.) were put into a reaction vessel and dissolved in DCM (30 mL) followed by stirring for 5 minutes, and then a solution in which tert-butyldiphenylsilyl chloride (TBDPS-Cl; 936.38 mg, 1 equiv.) was dissolved in DCM (20 mL) was added dropwise for 20 minutes. The liquid reaction mixture was stirred vigorously at room temperature for 5 hours. After confirming that the reaction was terminated via TLC (SiO₂; hexane/ethyl acetate, 19:1), the liquid reaction mixture was concentrated to a level of 35 mL. Afterwards, 70 mL of n-hexane was added to filter out precipitated solids, and the remaining filtrate was concentrated and purified by column chromatography (SiO₂; hexane/ethyl acetate, 39:1 to 19:1, v/v) to obtain Compound 105 (1183 mg, 90%) in clear liquid.

¹H NMR (CDCl₃, 400 MHz): δ 0.97 (t, J=6.2 Hz, 3H), 1.14 (s, 9H), 1.32 (s, 7H), 1.44-1.46 (m, 3H), 2.57 (s, 1H), 3.54-3.58 (m, 1H), 3.72-3.79 (m, 2H), 7.43-7.49 (m, 6H), 7.74 (d, J=6.7 Hz, 4H); ¹³C NMR (CDCl₃, 100 MHz): δ 14.15, 19.10, 22.65, 25.54, 26.92, 29.39, 31.82, 32.85, 68.12, 72.02, 127.83, 129.86, 129.86, 133.25, 133.29, 235.59, 135.61.

### 1-((tert-Butyldiphenylsilyl)oxy)octan-2-yl octanoate (106)

Compound 104 (100 mg, 1 equiv.) was put into in a reaction vessel and dissolved in DCM (50 mL), and then octanoic acid (62 mg, 1.3 equiv.), EDCI·HCl (46 mg, 1.5 equiv.), and DMAP (4 mg, 0.2 equiv.) were added to be subjected to reflux heating for 18 hours. After confirming that the reaction was terminated via TLC (SiO₂; hexane/ethyl acetate, 39:1), DCM (50 mL) was added additionally, followed by washing with distilled water (2×50 mL), saturated NaHCO₃ solution (2×50 mL), and brine (1×20 mL) sequentially. Organic layers were collected to remove moisture with anhydrous MgSO₄, the filtered filtrate was distilled under reduced pressure and purified by column chromatography (SiO₂; hexane/ethyl acetate, 39:1 to 19:1, v/v) to obtain Compound 106 (118 mg, 89%) in clear liquid.

¹H NMR (CDCl₃, 400 MHz): δ 0.98 (t, J=6.1 Hz, 6H), 1.14 (s, 9H), 1.22-1.33 (m, 16H), 1.47-1.55 (m, 4H), 2.17-2.22 (m, 2H), 3.55-3.63 (m, 2H), 4.90-4.95 (m, 1H), 7.27-7.36 (m, 6H), 7.57-7.59 (d, J=6.8 Hz, 4H); ¹³C NMR (CDCl₃, 100 MHz): δ 14.09, 19.26, 22.58, 22.63, 25.10, 25.16, 26.76, 29.00, 29.19, 30.56, 31.69, 31.72, 34.64, 65.09, 74.20, 127.67, 129.67, 129.66, 129.69, 133.53, 135.58, 135.65, 173.51.

### 1-Hydroxyoctan-2-yl octanoate (107)

After removing moisture from the reaction vessel by flame-drying, it was cooled to 20° C. and then filled with nitrogen gas. Thereafter, Compound 106 (200 mg, 1 equiv.) and dry THE (15 mL) were injected with a syringe and stirred for 1 minute. Thereafter, n-Bu₄NF solution (1M, THF; 0.5 mL, 1.2 equiv.) was added dropwise with a syringe for 10 minutes, and then the liquid reaction mixture was stirred at 20° C. for 2 hours. After confirming that the reaction was terminated via TLC (SiO₂; hexane/ethyl acetate, 9:1), distilled water (2×50 mL) was added for dilution, followed by extraction with DCM (3×40 mL). After organic layers were collected and washed with saturated NaCl solution, the moisture was removed with anhydrous Na₂SO₄, and a mixture obtained by distilling the filtered filtrate under reduced pressure was purified by column chromatography (SiO₂; hexane/ethyl acetate, 1:19, v/v) to obtain Compound 107 (96 mg, 90%) in colorless oil.

¹H NMR (CDCl₃, 400 MHz): δ 0.81 (t, J=6.3 Hz, 6H), 1.20 (m, 16H), 1.47-1.55 (m, 4H), 2.18-2.22 (m, 2H), 3.56-3.64 (m, 2H), 4.91-4.94 (m, 1H); ¹³C NMR (CDCl₃, 100 MHz): δ 14.09, 19.27, 22.54, 22.65, 25.14, 25.17, 26.88, 29.54, 30.56, 31.74, 31.72, 34.77, 65.09, 74.21, 173.56.

### 6-((2-(Octanoyloxy)octyl)oxy)-6-oxohexanoic acid (108)

Compound 107 was put into a reaction vessel and dissolved by adding DCM (25 mL), and then Compound 81 (84 mg, 2 equiv.) and DMAP (112 mg, 2.5 equiv.) were added and stirred vigorously at room temperature for 10 hours. After confirming that the reaction was terminated via TLC (SiO₂; hexane/ethyl acetate, 9:1), 1N aqueous HCl solution was added for acidification, followed by extraction with DCM (3×25 mL). After organic layers were collected and washed with saturated NaCl solution (1×30 mL), the moisture was removed with anhydrous Na₂SO₄, and a mixture obtained by distilling the filtered filtrate under reduced pressure was purified by column chromatography (SiO₂; hexane/ethyl acetate, 9:1 to 8:2, v/v) to obtain Compound 108 (99 mg, 70%) in clear liquid.

¹H NMR (CDCl₃, 400 MHz): δ 0.87 (t, J=6.9 Hz, 6H), 1.24-1.28 (m, 18H), 1.55-1.60 (m, 4H), 1.90-1.98 (m, 2H), 2.38 (t, J=6.1 Hz, 2H), 2.37-2.44 (m, 4H), 3.98-4.02 (m, 1H), 4.23 (dd, J=2.2, 9.1 Hz, 1H), 5.05-5.10 (m, 1H); ¹³C NMR (CDCl₃, 100 MHz): δ 14.12, 19.84, 22.48, 22.55, 24.84, 25.02, 28.87, 28.98, 29.01, 30.66, 31.56, 31.62, 32.90, 33.25, 32.90, 33.25, 34.05, 60.41, 64.81, 71.73, 172.37, 173.60, 178.67.

### 17-Hexyl-2,8,8-trimethyl-5,10,14-trioxo-6,15-dioxa-2,11-diazapentacosan-9-yl (2-(octanoyloxy)octyl) glutarate (Compound 12)

Compound 108 (170.90 mg, 1.2 equiv.) was dissolved in DCM (30 mL) in a reaction vessel, and then EDCI·HCl (113 mg, 1.6 equiv.) and DMAP (9 mg, 0.2 equiv.) were added, followed by stirring in the presence of argon gas at 5° C. for 10 minutes. A solution in which Compound 88 (200 mg, 1 equiv.) is dissolved in DCM (10 mL) was added dropwise at 5° C. and then stirred at room temperature for 20 hours. After confirming that the reaction was terminated via TLC (SiO₂; hexane/ethyl acetate, 7:3), DCM (50 mL) was added additionally, followed by washing with saturated NaHCO₃ solution (2×50 mL) and saturated NaCl solution (1×20 mL) sequentially. By removing moisture from an organic layer with anhydrous MgSO₄, filtrated filtered was distilled under reduced pressure and purified by column chromatography (SiO₂; ethyl acetate/hexane, 2:8 to 5:5, v/v) to obtain Compound 12 (292 mg, 87%) in clear liquid.

¹H NMR (CDCl₃, 400 MHz): δ 0.87 (t, J=6.5 Hz, 12H), 0.99 (d, J=4.7 Hz, 6H), 1.19 (m, 47H), 1.53 (m, 1H), 1.88-1.92 (m, 2H), 2.21 (s, 6H), 2.30-2.65 (m, 12H), 3.27-3.32 (m, 1H), 3.46-3.53 (m, 1H), 3.74 (d, J=11.2 Hz, 1H), 3.86-3.94 (m, 5H), 4.79 (s, 1H), 5.16 (br s, 1H), 7.35-7.38 (t, J=5.8 Hz, 1H); ¹³C NMR (CDCl₃, 100 MHz): δ 14.09, 20.05, 20.27, 22.04, 22.64, 22.66, 26.64, 26.69, 29.30, 29.56, 29.60, 29.94, 31.16, 31.21, 31.81, 31.88, 33.09, 33.24, 33.78, 35.04, 37.20, 37.24, 37.27, 45.21, 55.13, 67.33, 67.57, 69.59, 74.47, 76.61, 167.84, 171.69, 171.97, 172.43, 173.04, 176.04.

### [Scheme 6] Synthesis of Compound 13

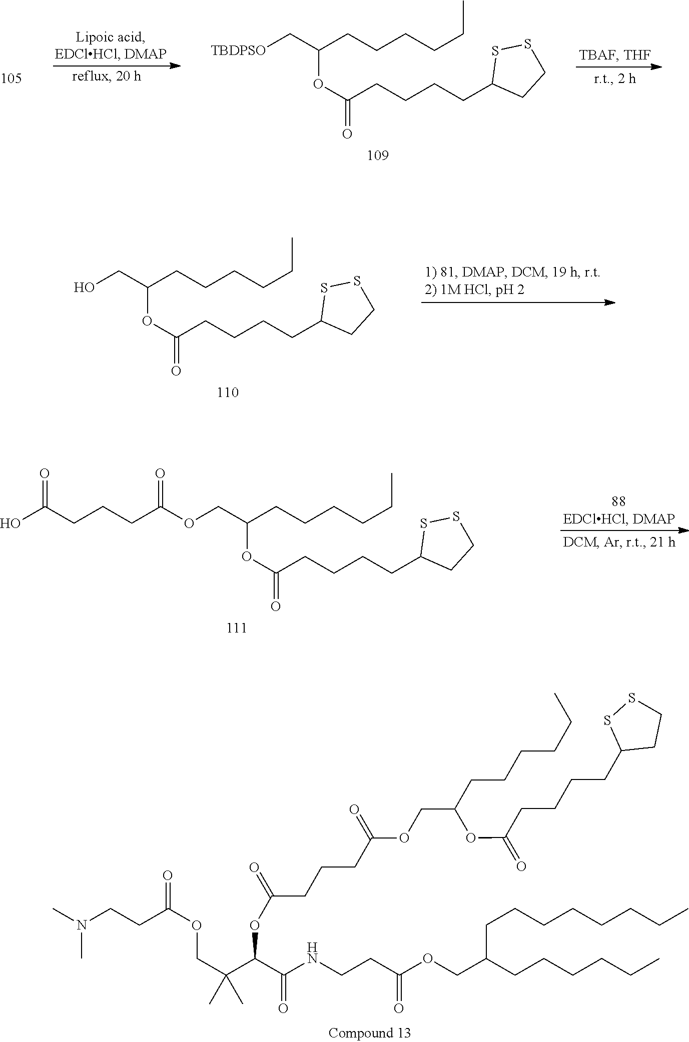

### 1-((tert-Butyldiphenylsilyl)oxy)octan-2-yl 5-(1,2-dithiolan-3-yl)pentanoate (109)

Compound 105 (500 mg, 1 equiv.) was put into a reaction vessel and dissolved in DCM (50 mL), lipoic acid (349 mg, 1.3 equiv.), EDCI·HCl (374 mg, 1.5 equiv.), EDCI·HCl (374 mg, 1.5 equiv.), and DMAP (31 mg, 0.2 equiv.) were added and subjected to reflux heating for 20 hours. After confirming that the reaction was terminated via TLC (SiO₂; hexane/ethyl acetate, 29:1), DCM (50 mL) was added additionally, and an organic layer was washed sequentially with distilled water (2×100 mL), saturated NaHCO₃ solution (2×100 mL), and saturated NaCl solution (1×40 mL). After distilling under reduced pressure, the filtrate filtered by removing moisture from the obtained organic layer with anhydrous MgSO₄, the obtained mixture was purified by column chromatography (SiO₂; hexane/ethyl acetate, 49:1 to 39:1, v/v) to obtain Compound 109 (603 mg, 81%) in yellow liquid.

¹H NMR (CDCl₃, 400 MHz): δ 0.87 (t, J=6.1 Hz, 3H), 1.04 (s, 9H), 1.25 (s, 8H), 1.44-1.66 (m, 8H), 1.68-1.89 (s, 1H), 2.26-2.31 (m, 2H), 2.42-2.43 (m, 1H), 3.09-3.16 (m, 2H), 3.51-3.55 (m, 1H), 3.66-3.68 (m, 2H), 5.00 (m, 1H), 7.25-7.42 (m, 6H), 7.65-7.66 (m, 4H); ¹³C NMR (CDCl₃, 100 MHz): δ 14.10, 19.27, 22.59, 24.79, 25.17, 26.78, 28.82, 29.18, 30.55, 31.71, 34.33, 34.33, 34.66, 38.49, 40.21, 56.12. 65.09, 74.38, 127.69, 129.69, 129.73, 133.45, 133.52, 135.57, 135.64, 173.10.

### 1-Hydroxyoctan-2-yl 5-(1,2-dithiolan-3-yl)pentanoate (110)

After removing moisture from the reaction vessel by flame-drying, it was cooled to 20° C. and filled with nitrogen gas. Thereafter, Compound 109 (600 mg, 1 equiv.) and dry THE (30 mL) were injected by a syringe and stirred for 1 minute. Subsequently, n-Bu₄NF solution (1M, THF; 1.26 mL, 1.2 equiv.) was added dropwise with a syringe for 10 minutes, and then the liquid reaction mixture was stirred at 20° C. for one hour. After confirming that the reaction was terminated via TLC (SiO₂; hexane/ethyl acetate, 8:2), distilled water (2×50 mL) was added for dilution, followed by extraction with DCM (3×40 mL). Organic layers were collected and washed with saturated NaCl solution, and then the mixture obtained by distilling under reduced pressure the filtrate filtered by removing moisture with anhydrous Na₂SO₄ was purified by column chromatography (SiO₂; hexane/ethyl acetate, 9:1, v/v) to obtain Compound 110 (297 mg, 85%) in yellow oil.

¹H NMR (CDCl₃, 400 MHz): δ 0.82 (t, J=6.7 Hz, 3H), 1.23-1.21 (m, 8H), 1.41-1.71 (m, 8H), 2.02 (m, 1H), 2.43-2.47 (m, 3H), 3.12-3.19 (m, 2H), 3.56 (qnt, J=6.7 Hz, 1H), 4.02-4.03 (m, 1H), 4.24 (dd, J=2.7, 12.0 Hz, 1H), 5.05 (m, 1H); ¹³C NMR (CDCl₃, 100 MHz): δ 14.11, 19.72, 22.73, 31.81, 25.32, 28.04, 29.02, 30.73, 32.71, 33.41, 34.24, 34.63, 38.55, 40.33, 56.34, 65.56, 70.73, 173.14, 173.65, 178.45.

### 5-((2-((5-(1,2-Dithiolan-3-yl)pentanoyl)oxy)octyl)oxy)-5-oxopentanoic acid (111)

In the reaction vessel, Compound 81 (204 mg, 2 equiv.) and DMAP (274 mg, 2.5 equiv.) were added to a solution, in which Compound 110 (300 mg, 1 equiv.) and DCM (25 mL) were added, followed by stirring at room temperature. After vigorously stirring the reaction mixture at room temperature for 19 hours and then checking that the reaction was terminated via TLC (SiO₂; hexane/ethyl acetate, 8:2), acidification was followed with 1M aqueous HCl solution. The obtained liquid mixture was extracted with DCM (3×50 mL) to collect organic layers, followed by washing with aqueous saturated NaCl solution (1×50 mL), and then the mixture obtained by distilling under reduced pressure the filtrate filtered by removing moisture with anhydrous Na₂SO₄ was purified by column chromatography (SiO₂; hexane/ethyl acetate, 9:1 to 8:2, v/v) to obtain Compound 111 (302 mg, 75%) in yellow liquid.

¹H NMR (CDCl₃, 400 MHz): δ 0.88 (t, J=6.7 Hz, 3H), 1.27-1.29 (m, 8H), 1.45-1.70 (m, 8H), 1.88-2.02 (m, 5H), 2.35 (t, J=8.0 Hz, 2H), 2.41-2.48 (m, 8H), 3.11-3.18 (m, 2H), 3.55-3.58 (qnt, J=6.8 Hz, 1H), 3.98-4.03 (m, 1H), 4.26 (dd, J=2.7, 11.9 Hz, 1H), 5.09 (m, 1H); ¹³C NMR (CDCl₃, 100 MHz): δ 14.10, 19.72, 22.79, 31.79, 29.31, 25.32, 28.01, 29.03, 30.72, 32.75, 33.31, 34.23, 34.62, 38.51, 40.20, 56.33, 65.55, 70.71, 173.11, 173.60, 178.43.

### 2-((5-(1,2-Dithiolan-3-yl)pentanoyl)oxy)octyl (17-hexyl-2,8,8-trimethyl-5,10,14-trioxo-6,15-dioxa-2,11-diazapentacosan-9-yl) glutarate (Compound 13)

Compound 111 (215 mg, 1.3 equiv.) was put into a reaction vessel and dissolved in DCM (30 mL), and then EDCI·HCl (113 mg, 1.6 equiv.) and DMAP (9 mg, 0.2 equiv.) were added and stirred vigorously in the presence of argon gas at 5° C. for 10 minutes. A solution in which Compound 88 (200 mg, 1 equiv.) is dissolved in DCM (10 mL) was added dropwise at 5° C. and stirred at room temperature for 21 hours. After confirming that the reaction was terminated via TLC (SiO₂; ethyl acetate), DCM (50 mL) was added additionally, followed by washing with anhydrous saturated NaHCO₃ solution (2×50 mL) and saturated NaCl solution (1×20 mL) sequentially. After removing moisture from an organic layer with anhydrous MgSO₄, and then the filtered filtrate was distilled under reduced pressure and purified by column chromatography (SiO₂; ethyl acetate/hexane, 5:5 to 7:3, v/v) to obtain Compound 13 (251 mg, 70%) in yellow liquid.

¹H NMR (CDCl₃, 400 MHz): δ 0.88 (t, J=6.9 Hz, 9H), 1.06 (d, J=6.5 Hz, 6H), 1.26 (m, 33H), 1.45-1.71 (m, 10H), 1.90-1.99 (m, 3H), 2.28 (s, 6H), 2.32 (t, J=7.4 Hz, 2H), 2.40 (t, J=7.2 Hz, 2H), 2.44-2.61 (m, 8H), 2.67-2.72 (m, 1H), 3.08-3.21 (m, 2H), 3.34-3.39 (m, 1H), 3.53-3.60 (m, 2H), 3.81 (d, J=11.2 Hz, 1H), 3.96-4.04 (m, 4H), 4.23-4.26 (m, 1H), 4.85 (s, 1H), 5.06-5.09 (m, 1H), 7.45 (t, J=5.7 Hz, 1H); ¹³C NMR (CDCl₃, 100 MHz): δ 14.01, 14.08, 14.09, 20.04, 20.26, 22.02, 22.51, 22.63, 22.65, 24.56, 25.06, 26.63, 26.67, 28.69, 29.00, 29.29, 29.54, 29.58, 29.92, 30.71, 31.15, 31.59, 31.79, 31.87, 33.02, 33.04, 33.29, 33.79, 33.83, 34.57, 35.05, 37.19, 37.23, 38.46, 40.20, 45.19, 55.10, 56.30, 64.94, 67.57, 67.57, 69.57, 71.69, 76.61, 167.81, 171.66, 171.92, 172.35, 172.39, 173.12.

### <Preparation Example 3> Preparation of Helper Lipids, Structure-Maintaining Lipids, and PEG-Modified Lipids

1,2-Dioleoyl-sn-glycero-3-phosphoethanolamine (DOPE) and 1,2-distearoyl-sn-glycero-3-phophocholine (DSPC) were purchased from Sigma-Aldrich for preparation as helper lipids, and trehalose-based lipids were synthesized by the following methods.

After dissolving trehalose dihydrate (200 mg, 0.53 mmol) in pyridine (5 mL), TBTU (421 mg, 1.3 mmol), DIPEA (320 μL, 1.2 mmol), and oleic acid (370 μL, 1.2 mmol) were added in order, and after filling with argon gas, the mixture was stirred at room temperature for 20 hours. The solvent was removed using a rotary reduced pressure distiller, and then dried after primary purification by column chromatography (SiO₂, MeOH/EtOAc 2:98->10:90). After washing the dried solids several times with EtOAc, it was completely dried to obtain 287 mg (62%) of 6,6′-trehalose dioleate.

¹H NMR (400 MHz, CD₃OD) δ 5.38 (t, J=4.5 Hz, 4H), 5.08 (d, J=3.8 Hz, 2H), 4.39 (dd, J=2.2, 11.9 Hz, 2H), 4.23 (dd, J=5.1, 11.9 Hz, 2H), 4.04 (ddd, J=2.1, 5.3, 10.2 Hz, 2H), 3.81 (dd, J=9.4, 9.6 Hz, 2H), 3.50 (dd, J=3.6, 9.6 Hz, 2H), 3.35 (dd, J=8.8, 10.0 Hz, 2H), 2.04-2.09 (m, 8H), 2.37 (t, J=7.4 Hz, 4H), 1.61-1.67 (m, 4H), 1.29-1.41 (m, 40H), 0.93 (t, J=6.8 Hz, 6H); ¹³C NMR (100 MHz, CD₃OD) δ 174.0, 129.5, 129.4, 93.8, 73.2, 71.8, 70.5, 70.1, 63.0, 33.7, 31.7, 29.5, 29.4, 29.2, 29.1, 29.0, 28.9, 28.8, 28.8, 26.8, 24.7, 22.4, 13.1.

Cholesterol and butyl lithocholate were prepared as the structure-maintaining lipid, lytocholic acid derivative compounds were synthesized by the following methods, and myristoyl diglyceride (DMG)-PEG was purchased and prepared as the PEG-modified lipid.

Lithocholic acid and n-butanol were put into a reaction vessel, and then HCl solution (35˜37% aqueous solution, 1 equiv.) was injected, followed by stirring at room temperature for 24 hours. After distilling the alcohol using a rotary reduced pressure distiller, it was dissolved again in DCM to extract impurities with saturated NaHCO₃ aqueous solution and saturated brine. After that, the moisture was removed from the DCM layer using anhydrous Na₂SO₄ followed by filtration, and the filtered solution was dried using a rotary reduced pressure distiller and purified by column chromatography (SiO₂, DCM/MeOH, 19:1˜49:1 volume ratio) to obtain a lithocholic acid derivative compound (92%).

MS (ESI-MS) calcd. for C₂₈H₄₆NO₂ [M+H]+ 428.3523, found 428.3525.

### <Example 1> Fabrication of Nucleic Acid Molecules of the RNA Platform

Fabricated was an RNA platform that is inserted with a nucleic acid sequence as a target sequence that encodes Renilla Luciferase (R/L) which is known as one of the reporter genes while having internal ribosome entry site (IRES) element derived from encephalomyocarditis virus (EMCV). The template DNA was designed, and single-stranded nucleic acid molecules for the RNA platform were fabricated using in vitro transcription (IVT).

### <Example 2> Preparation of Lipid Nanoparticles

After preparing the lipid nanoparticle with RNA solution (50 mM sodium citrate buffer, 110 mM NaCl, pH=4.0) in a concentration of 0.625 mg/mL and a lipid mixture solution (ethanol) in Table 1 below using laboratory mixer and emulsifier (Namoassemblr Spark, Precision Nanosystems, Inc.), it was provided by undergoing solvent conversion with normal saline or PBS using a filter tube (UFC5010, Amicon) for centrifugation.

TABLE 1

| | Ionizable | | Structure-maintaining | | Gallic acid |
|---|---|---|---|---|---|
| | lipid | Helper lipid | lipid | PEG-modified | derivative |
| | (mol %) | (mol %) | (mol %) | lipid (mol %) | (mol %) |
| LNP 126 | SM-102 | DOPE | Butyl lithocholate | DMG-PEG | — |
| | (25) | (10) | (38.5) | (1.5) | |
| | | 6,6′-trehalose | | | |
| | | dioleate (25) | | | |
| LNP 128 | SM-102 | DSPC | Cholesterol | DMG-PEG | — |
| | (50) | (10) | (38.5) | (1.5) | |
| LNP 201a | SM-102 | DOPE | Butyl lithocholate | DMG-PEG | Chemical |
| | (37.5) | (10) | (38.5) | (1.5) | Formula 6 |
| | | | | | (12.5) |
| LNP 201b | SM-102 | DOPE | Butyl lithocholate | DMG-PEG | Chemical |
| | (25) | (10) | (38.5) | (1.5) | Formula 6 |
| | | | | | (25) |
| LNP 201c | SM-102 | DOPE | Butyl lithocholate | DMG-PEG | Chemical |
| | (12.5) | (10) | (38.5) | (1.5) | Formula 6 |
| | | | | | (37.5) |
| LNP 201d | SM-102 | DOPE | Butyl lithocholate | DMG-PEG | Chemical |
| | (0) | (10) | (38.5) | (1.5) | Formula 6 |
| | | | | | (50) |
| LNP 218 | SM-102 | DOPE | Butyl lithocholate | DMG-PEG | Chemical |
| | (25) | (10) | (38.5) | (1.5) | Formula 2 |
| | | | | | (25) |
| LNP 219 | SM-102 | DOPE | Butyl lithocholate | DMG-PEG | Chemical |
| | (25) | (10) | (38.5) | (1.5) | Formula 3 |
| | | | | | (25) |
| LNP 220 | SM-102 | DOPE | Butyl lithocholate | DMG-PEG | Chemical |
| | (25) | (10) | (38.5) | (1.5) | Formula 4 |
| | | | | | (25) |
| LNP 221 | SM-102 | DOPE | Butyl lithocholate | DMG-PEG | Chemical |
| | (25) | (10) | (38.5) | (1.5) | Formula 5 |
| | | | | | (25) |
| LNP 222 | SM-102 | DOPE | — | DMG-PEG | Chemical |
| | (25) | (10) | | (1.5) | Formula 2 |
| | | 6,6′-trehalose | | | (38.5) |
| | | dioleate (25) | | | |
| LNP 223 | SM-102 | DOPE | — | DMG-PEG | Chemical |
| | (25) | (10) | | (1.5) | Formula 3 |
| | | 6,6′-trehalose | | | (38.5) |
| | | dioleate (25) | | | |
| LNP 284 | Chemical | DOPE | Butyl lithocholate | DMG-PEG | Chemical |
| | Formula 8 | (10) | (38.5) | (1.5) | Formula 2 |
| | (25) | | | | (25) |
| LNP 285 | Chemical | DOPE | Butyl lithocholate | DMG-PEG | Chemical |
| | Formula 8 | (10) | (38.5) | (1.5) | Formula 3 |
| | (25) | | | | (25) |
| LNP 286 | Chemical | DOPE | Butyl lithocholate | DMG-PEG | Chemical |
| | Formula 8 | (10) | (38.5) | (1.5) | Formula 4 |
| | (25) | | | | (25) |
| LNP 287 | Chemical | DOPE | Butyl lithocholate | DMG-PEG | Chemical |
| | Formula 8 | (10) | (38.5) | (1.5) | Formula 5 |
| | (25) | | | | (25) |
| LNP 288 | Chemical | DOPE | — | DMG-PEG | Chemical |
| | Formula 8 | (10) | | (1.5) | Formula 2 |
| | (25) | 6,6′-trehalose | | | (38.5) |
| | | dioleate (25) | | | |
| LNP 289 | Chemical | DOPE | — | DMG-PEG | Chemical |
| | Formula 8 | (10) | | (1.5) | Formula 3 |
| | (25) | 6,6′-trehalose | | | (38.5) |
| | | dioleate (25) | | | |
| LNP 297 | Chemical | DOPE | Butyl lithocholate | DMG-PEG | Chemical |
| | Formula 9 | (10) | (38.5) | (1.5) | Formula 2 |
| | (25) | | | | (25) |
| LNP 298 | Chemical | DOPE | Butyl lithocholate | DMG-PEG | Chemical |
| | Formula 9 | (10) | (38.5) | (1.5) | Formula 3 |
| | (25) | | | | (25) |
| LNP 299 | Chemical | DOPE | Butyl lithocholate | DMG-PEG | Chemical |
| | Formula 9 | (10) | (38.5) | (1.5) | Formula 4 |
| | (25) | | | | (25) |
| LNP 300 | Chemical | DOPE | Butyl lithocholate | DMG-PEG | Chemical |
| | Formula 9 | (10) | (38.5) | (1.5) | Formula 5 |
| | (25) | | | | (25) |
| LNP 301 | Chemical | DOPE | — | DMG-PEG | Chemical |
| | Formula 9 | (10) | | (1.5) | Formula 2 |
| | (25) | 6,6′-trehalose | | | (38.5) |
| | | dioleate (25) | | | |
| LNP 302 | Chemical | DOPE | — | DMG-PEG | Chemical |
| | Formula 9 | (10) | | (1.5) | Formula 3 |
| | (25) | 6,6′-trehalose | | | (38.5) |
| | | dioleate (25) | | | |
| LNP 316 | SM-102 | DOPE | — | DMG-PEG | Chemical |
| | (25) | (10) | | (1.5) | Formula 4 |
| | | 6,6′-trehalose | | | (38.5) |
| | | dioleate (25) | | | |
| LNP 317 | SM-102 | DOPE | — | DMG-PEG | Chemical |
| | (25) | (10) | | (1.5) | Formula 5 |
| | | 6,6′-trehalose | | | (38.5) |
| | | dioleate (25) | | | |
| LNP 318 | Chemical | DOPE | — | DMG-PEG | Chemical |
| | Formula 8 | (10) | | (1.5) | Formula 4 |
| | (25) | 6,6′-trehalose | | | (38.5) |
| | | dioleate (25) | | | |
| LNP 319 | Chemical | DOPE | — | DMG-PEG | Chemical |
| | Formula 8 | (10) | | (1.5) | Formula 5 |
| | (25) | 6,6′-trehalose | | | (38.5) |
| | | dioleate (25) | | | |
| LNP 320 | Chemical | DOPE | — | DMG-PEG | Chemical |
| | Formula 9 | (10) | | (1.5) | Formula 4 |
| | (25) | 6,6′-trehalose | | | (38.5) |
| | | dioleate (25) | | | |
| LNP 321 | Chemical | DOPE | — | DMG-PEG | Chemical |
| | Formula 9 | (10) | | (1.5) | Formula 5 |
| | (25) | 6,6′-trehalose | | | (38.5) |
| | | dioleate (25) | | | |
| LNP 339 | Chemical | DOPE | Butyl lithocholate | DMG-PEG | Chemical |
| | Formula 10 | (10) | (38.5) | (1.5) | Formula 2 |
| | (25) | | | | (25) |
| LNP 340 | Chemical | DOPE | Butyl lithocholate | DMG-PEG | Chemical |
| | Formula 10 | (10) | (38.5) | (1.5) | Formula 3 |
| | (25) | | | | (25) |
| LNP 341 | Chemical | DOPE | Butyl lithocholate | DMG-PEG | Chemical |
| | Formula 10 | (10) | (38.5) | (1.5) | Formula 4 |
| | (25) | | | | (25) |
| LNP 342 | Chemical | DOPE | Butyl lithocholate | DMG-PEG | Chemical |
| | Formula 10 | (10) | (38.5) | (1.5) | Formula 5 |
| | (25) | | | | (25) |
| LNP 343 | Chemical | DOPE | — | DMG-PEG | Chemical |
| | Formula 10 | (10) | | (1.5) | Formula 2 |
| | (25) | 6,6′-trehalose | | | (38.5) |
| | | dioleate (25) | | | |
| LNP 344 | Chemical | DOPE | — | DMG-PEG | Chemical |
| | Formula 10 | (10) | | (1.5) | Formula 3 |
| | (25) | 6,6′-trehalose | | | (38.5) |
| | | dioleate (25) | | | |
| LNP 347 | Chemical | DOPE | — | DMG-PEG | Chemical |
| | Formula 10 | (10) | | (1.5) | Formula 4 |
| | (25) | 6,6′-trehalose | | | (38.5) |
| | | dioleate (25) | | | |
| LNP 348 | Chemical | DOPE | — | DMG-PEG | Chemical |
| | Formula 10 | (10) | | (1.5) | Formula 5 |
| | (25) | 6,6′-trehalose | | | (38.5) |
| | | dioleate (25) | | | |
| LNP 349 | SM-102 | DSPC | — | DMG-PEG | Chemical |
| | (50) | (10) | | (1.5) | Formula 2 |
| | | | | | (38.5) |
| LNP 350 | SM-102 | DSPC | — | DMG-PEG | Chemical |
| | (50) | (10) | | (1.5) | Formula 3 |
| | | | | | (38.5) |
| LNP 351 | SM-102 | DSPC | — | DMG-PEG | Chemical |
| | (50) | (10) | | (1.5) | Formula 4 |
| | | | | | (38.5) |
| LNP 352 | SM-102 | DSPC | — | DMG-PEG | Chemical |
| | (50) | (10) | | (1.5) | Formula 5 |
| | | | | | (38.5) |
| LNP 353 | Chemical | DSPC | — | DMG-PEG | Chemical |
| | Formula 8 | (10) | | (1.5) | Formula 2 |
| | (50) | | | | (38.5) |
| LNP 354 | Chemical | DSPC | — | DMG-PEG | Chemical |
| | Formula 8 | (10) | | (1.5) | Formula 3 |
| | (50) | | | | (38.5) |
| LNP 355 | Chemical | DSPC | — | DMG-PEG | Chemical |
| | Formula 8 | (10) | | (1.5) | Formula 4 |
| | (50) | | | | (38.5) |
| LNP 356 | Chemical | DSPC | — | DMG-PEG | Chemical |
| | Formula 8 | (10) | | (1.5) | Formula 5 |
| | (50) | | | | (38.5) |
| LNP 357 | Chemical | DSPC | — | DMG-PEG | Chemical |
| | Formula 9 | (10) | | (1.5) | Formula 2 |
| | (50) | | | | (38.5) |
| LNP 358 | Chemical | DSPC | — | DMG-PEG | Chemical |
| | Formula 9 | (10) | | (1.5) | Formula 3 |
| | (50) | | | | (38.5) |
| LNP 359 | Chemical | DSPC | — | DMG-PEG | Chemical |
| | Formula 9 | (10) | | (1.5) | Formula 4 |
| | (50) | | | | (38.5) |
| LNP 360 | Chemical | DSPC | — | DMG-PEG | Chemical |
| | Formula 9 | (10) | | (1.5) | Formula 5 |
| | (50) | | | | (38.5) |
| LNP 361 | Chemical | DSPC | — | DMG-PEG | Chemical |
| | Formula 11 | (10) | | (1.5) | Formula 2 |
| | (50) | | | | (38.5) |
| LNP 362 | Chemical | DSPC | — | DMG-PEG | Chemical |
| | Formula 11 | (10) | | (1.5) | Formula 3 |
| | (50) | | | | (38.5) |
| LNP 363 | Chemical | DSPC | — | DMG-PEG | Chemical |
| | Formula 11 | (10) | | (1.5) | Formula 4 |
| | (50) | | | | (38.5) |
| LNP 364 | Chemical | DSPC | — | DMG-PEG | Chemical |
| | Formula 11 | (10) | | (1.5) | Formula 5 |
| | (50) | | | | (38.5) |
| LNP 412 | Chemical | DSPC | Cholesterol | DMG-PEG | — |
| | Formula 12 | (10) | (38.5) | (1.5) | |
| | (50) | | | | |
| LNP 413 | Chemical | DSPC | — | DMG-PEG | Chemical |
| | Formula 12 | (10) | | (1.5) | Formula 3 |
| | (50) | | | | (38.5) |
| LNP 414 | Chemical | DSPC | — | DMG-PEG | Chemical |
| | Formula 12 | (10) | | (1.5) | Formula 5 |
| | (50) | | | | (38.5) |
| LNP 415 | Chemical | DOPE | Butyl lithocholate | DMG-PEG | Chemical |
| | Formula 12 | (10) | (38.5) | (1.5) | Formula 3 |
| | (25) | | | | (25) |
| LNP 416 | Chemical | DOPE | Butyl lithocholate | DMG-PEG | Chemical |
| | Formula 12 | (10) | (38.5) | (1.5) | Formula 5 |
| | (25) | | | | (25) |
| LNP 417 | Chemical | DSPC | Cholesterol | DMG-PEG | — |
| | Formula 13 | (10) | (38.5) | (1.5) | |
| | (50) | | | | |
| LNP 418 | Chemical | DSPC | — | DMG-PEG | Chemical |
| | Formula 13 | (10) | | (1.5) | Formula 3 |
| | (50) | | | | (38.5) |
| LNP 419 | Chemical | DSPC | — | DMG-PEG | Chemical |
| | Formula 13 | (10) | | (1.5) | Formula 5 |
| | (50) | | | | (38.5) |
| LNP 420 | Chemical | DOPE | Butyl lithocholate | DMG-PEG | Chemical |
| | Formula 15 | (10) | (38.5) | (1.5) | Formula 3 |
| | (25) | | | | (25) |
| LNP 421 | Chemical | DOPE | Butyl lithocholate | DMG-PEG | Chemical |
| | Formula 15 | (10) | (38.5) | (1.5) | Formula 5 |
| | (25) | | | | (25) |

### <Experimental Example 1> Evaluation of a Delivery Efficiency by Identifying the Protein Expression Level

Expression was identified by intradermal injection (ID) of mRNA-LNP into the ears of ICR mice at 5 g/20 μL based on RNA.

The experimental process is as follows. Each experimental group was prepared at a dose of 5 g/20 μL based on RNA, and then injected via I.D. into both ears of the mouse, respectively. Afterwards, the expression was checked according to the maximum expression time band of target proteins. In the case of Renilla Luciferase (R/L), which was actually tested, the expression and maintenance were checked according to two time bands which are 6 hours and 24 hours.

The specific experimental process is as follows.

After anesthesia using an individual respiratory anesthesia machine, the drug administered according to the experimental conditions was injected via an insulin syringe. Afterwards, anesthesia was conducted using CO₂ at each corresponding time, and then ears were cut off. The cut ears were completely crushed using scissors and a homogenizer while being placed in 300 μL of 1× Renilla Lysis buffer. Afterwards, 20 μL of the liquid mixture was taken and transferred to a white 96-well plate, 100 μL of Promega's Renilla luciferase assay substrate was added, and then luminescence was measured to compare the expression.

### <Experimental Example 2> Identification of the Expression Level of the MCP-1 Protein Increased by R/L Encoding mRNA-Formulated LNP

Monocyte chemoattractant protein-1 (MCP-1) is a type of chemokine that causes monocytes to migrate toward vascular endothelial cells for attachment and is known to play an important role in the immune response. In order to identify the effect of LNP on the immune response, mRNA encoding R/L was encapsulated into the LNP, then mRNA-LNP was injected via I.D. into the ears of ICR mice at 5 g/20 μL based on RNA, and blood was collected 6 hours later, followed by concentration measurement through ELISA.

The specific experimental process is as follows.

The drug administered according to the experimental conditions was injected through an insulin syringe. After 6 hours, blood was drawn using a respiratory anesthesia machine. After separating the serum from the secured blood, the experiment was carried out. Expression levels were compared using Invitrogen's MCP-1 mouse uncoated ELISA kit.

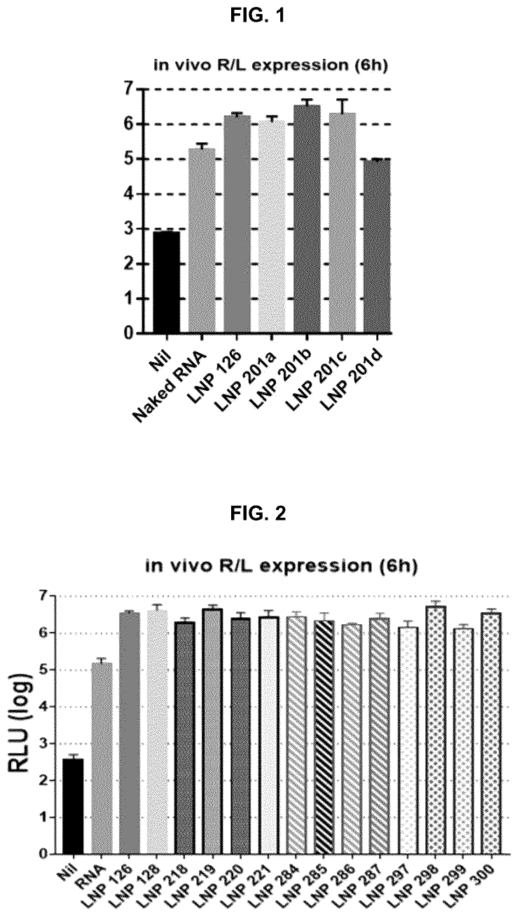

FIG. 1 shows a result of analyzing a protein expression efficiency by measuring an amount of R/L expressed 6 hours after administering lipid nanoparticles (LNP 201a, 201b, 201c, 201d), which is in a composition having a gallic acid derivative that includes Renilla Luciferase (R/L) mRNA, and positive control lipid nanoparticles (LNP 126) into the ears of ICR mice via I.D.

In FIG. 1, when the ratio of SM-102 to Chemical Formula 6 was formulated as 37.5/12.5 (LNP 201a), 25/25 (LNP 201b), 12.5/37.5 (LNP 201c), and 0/50 (LNP 201d) by substituting 6,6′-trehalose dioleate of LNP 126 with Chemical Formula 6, the protein expression efficiency was maintained or significantly increased, except for LNP 201d. In particular, the protein expression efficiency of LNP 202b increased significantly, while that of LNP 201d, which contains no ionizable lipid, decreased significantly.

FIG. 2 shows a result of analyzing a protein expression efficiency by measuring an amount of R/L expressed 6 hours after administering lipid nanoparticles (LNP 218, 219, 220, 221, 284, 285, 286, 287, 297, 298, 299, 300), which is in a composition having a gallic acid derivative that includes Renilla Luciferase (R/L) mRNA, and positive control lipid nanoparticles (LNP 126, 128) into the ears of ICR mice via I.D.

In FIG. 2, when 6,6′-trehalose dioleate of LNP 126 was substituted by propyl gallate (LNP 218), dodecyl gallate (LNP 219), hexadecyl gallate (LNP 220), and octadecyl gallate (LNP 221), respectively, or when SM-102 of LNP 126 was substituted by Chemical Formula 8 and 6,6′-trehalose dioleate was substituted by propyl gallate (LNP 284), dodecyl gallate (LNP 285), hexadecyl gallate (LNP 286), and octadecyl gallate (LNP 287), respectively, or when SM-102 of LNP 126 was substituted by Chemical Formula 9 and 6,6′-trehalose dioleate was substituted by propyl gallate (LNP 297), dodecyl gallate (LNP 298), hexadecyl gallate (LNP 299), and octadecyl gallate (LNP 300), respectively, the protein expression efficiency remained approximately at a similar level. When optimized with gallic acid derivatives using Chemical Formula 9, the protein expression efficiency increased significantly when dodecyl gallate (LNP 298) was used.

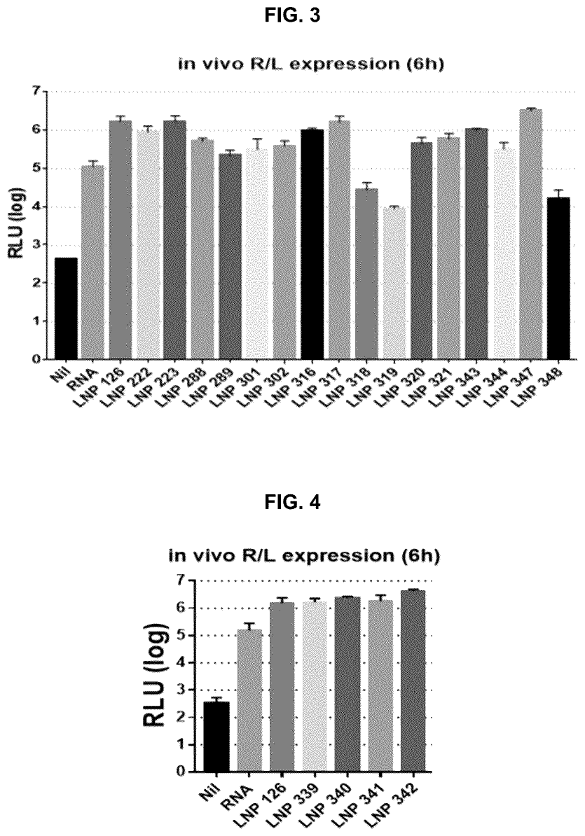

FIG. 3 shows a result of analyzing a protein expression efficiency by measuring an amount of R/L expressed 6 hours after administering lipid nanoparticles (LNP 222, 223, 288, 289, 301, 302, 316, 317, 318, 319, 320, 321, 343, 344, 347, 348), which is in a composition having a gallic acid derivative that includes Renilla Luciferase (R/L) mRNA, and positive control lipid nanoparticles (LNP 126) into the ears of ICR mice via I.D.

In FIG. 3, when butyl lithocholate of LNP 126 was substituted by propyl gallate (LNP 222), dodecyl gallate (LNP 223), hexadecyl gallate (LNP 316), and octadecyl gallate (LNP 317), respectively, or when SM-102 of LNP 126 was substituted by Chemical Formula 8 and butyl lithocholate was substituted by propyl gallate (LNP 288), dodecyl gallate (LNP 289), hexadecyl gallate (LNP 318), and octadecyl gallate (LNP 319), respectively, or when SM-102 of LNP 126 was substituted by Chemical Formula 9 and butyl lithocholate was substituted by propyl gallate (LNP 301), dodecyl gallate (LNP 302), hexadecyl gallate (LNP 320), and octadecyl gallate (LNP 321), respectively, or when SM-102 of LNP 126 was substituted by Chemical Formula 10 and butyl lithocholate was substituted by propyl gallate (LNP 343), dodecyl gallate (LNP 344), hexadecyl gallate (LNP 347), and octadecyl gallate (LNP 348), respectively, the protein expression efficiency remained approximately at a similar level or increased or decreased significantly. When optimized with gallic acid derivatives using Chemical Formula 10, the protein expression efficiency increased significantly, especially when hexadecyl gallate (LNP 347) was used. LNP 223 and LNP 317 maintained the protein expression efficiency similar to those of positive controls.

FIG. 4 shows a result of analyzing a protein expression efficiency by measuring an amount of R/L expressed 6 hours after administering lipid nanoparticles (LNP 339, 340, 341, 342), which is in a composition having a gallic acid derivative that includes Renilla Luciferase (R/L) mRNA, and positive control lipid nanoparticles (LNP 126) into the ears of ICR mice via I.D.

In FIG. 4, when SM-102 of LNP 126 was substituted by Chemical Formula 10 and 6,6′-trehalose dioleate was substituted by propyl gallate (LNP 339), dodecyl gallate (LNP 340), hexadecyl gallate (LNP 341), and octadecyl gallate (LNP 342), respectively, the protein expression efficiency remained approximately at the same level or increased significantly, except for LNP 347. When optimized with gallic acid derivatives using Chemical Formula 10, the protein expression efficiency increased significantly, especially when octadecyl gallate (LNP 342) was used.

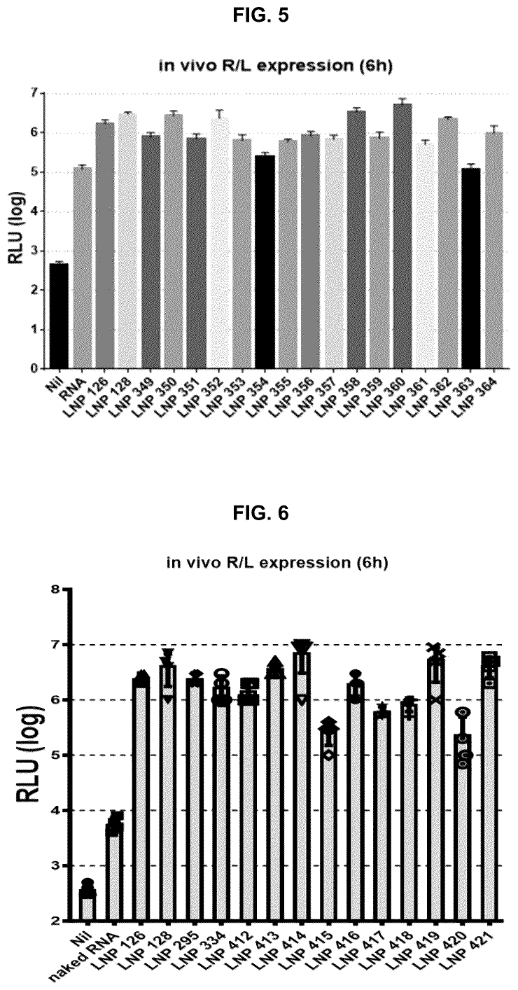

FIG. 5 shows a result of analyzing a protein expression efficiency by measuring an amount of R/L expressed 6 hours after injecting lipid nanoparticles (LNP 349, 350, 351, 352, 353, 354, 355, 356, 357, 358, 359, 360, 361, 362, 363, 364), which is in a composition having a gallic acid derivative that includes Renilla Luciferase (R/L) mRNA, and positive control lipid nanoparticles (LNP 126, 128) into the ears of ICR mice.

In FIG. 5, when cholesterol of LNP 128 was substituted by propyl gallate (LNP 349), dodecyl gallate (LNP 350), hexadecyl gallate (LNP 351), and octadecyl gallate (LNP 352), respectively, or when SM-102 of LNP 128 was substituted by Chemical Formula 8 and cholesterol was substituted by propyl gallate (LNP 353), dodecyl gallate (LNP 354), hexadecyl gallate (LNP 355), and octadecyl gallate (LNP 356), respectively, or when SM-102 of LNP 128 was substituted by Chemical Formula 9 and cholesterol was substituted by propyl gallate (LNP 357), dodecyl gallate (LNP 358), hexadecyl gallate (LNP 359), and octadecyl gallate (LNP 360), respectively, or when SM-102 of LNP 128 was substituted by Chemical Formula 11 and cholesterol was substituted by propyl gallate (LNP 361), dodecyl gallate (LNP 362), hexadecyl gallate (LNP 363), and octadecyl gallate (LNP 364), respectively, the protein expression efficiency remained approximately at the same level or significantly decreased, except for LNP 360. When optimized with gallic acid derivatives using Chemical Formula 9, the protein expression efficiency increased significantly, especially when octadecyl gallate (LNP 360) was used. LNP 350, LNP 352, LNP358, and LNP362 maintained the protein expression efficiency similar to those of positive controls.

FIG. 6 shows a result of analyzing a protein expression efficiency by measuring an amount of R/L expressed 6 hours after administering lipid nanoparticles (LNP 412, 413, 414, 415, 416, 417, 418, 419, 420, 421), which is in a composition having a gallic acid derivative that includes Renilla Luciferase (R/L) mRNA, and positive control lipid nanoparticles (LNP 126, 128, 295, 334) into the ears of ICR mice via I.D.

In FIG. 6, when SM-102 of LNP 128 was substituted by Chemical Formula 12 (LNP 412) or when cholesterol of LNP 412 was substituted by dodecyl gallate (LNP 413) and octadecyl gallate (LNP 414), respectively, or when 6,6′-trehalose dioleate of LNP 412 was substituted by dodecyl gallate (LNP 415) and octadecyl gallate (LNP 416), respectively, or when SM-102 of LNP 128 was substituted by Chemical Formula 13 (LNP 417) or cholesterol of LNP 417 was substituted by dodecyl gallate (LNP 418) and octadecyl gallate (LNP 419), respectively, or when 6,6′-trehalose dioleate of LNP 417 was substituted by dodecyl gallate (LNP 420) and octadecyl gallate (LNP 421), respectively, the protein expression efficiency remained approximately at the same level or significantly increased or decreased. When optimized with gallic acid derivatives using Chemical Formula 12, the protein expression efficiency increased significantly, especially when octadecyl gallate (LNP 414) was used instead of cholesterol. When optimized with gallic acid derivatives using Chemical Formula 13, the protein expression efficiency increased significantly, especially when octadecyl gallate (LNP 419) was used instead of 6,6′-trehalose dioleate as well as octadecyl gallate (LNP 421) was used instead of cholesterol. In particular, the protein expression efficiency was significantly reduced when Chemical Formula 13 was used as an ionizable lipid (LNP 417) instead of SM-102 in LNP 128, but the protein expression efficiency was significantly increased when octadecyl gallate (LNP 419) was used instead of cholesterol of LNP 417.

Referring to FIG. 2 to FIG. 6, the protein expression efficiency of LNP tends to increase as the number of carbons of the alkyl group introduced into the gallic acid derivative increases, and the protein expression efficiency of LNP347 with hexadecyl gallate introduced and LNP 342, LNP 360, LNP 414, LNP 419, and LNP 421 with octadecyl gallate introduced particularly increased.

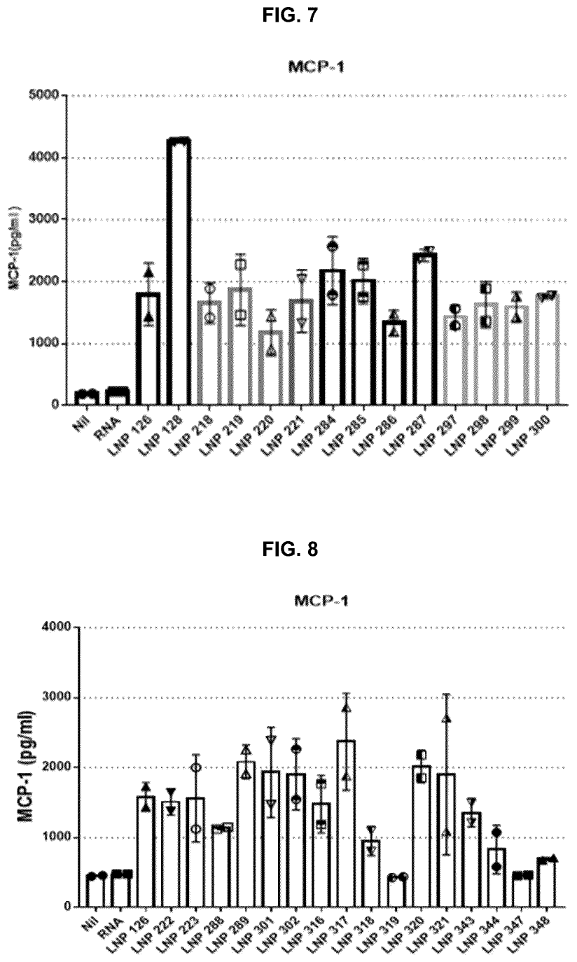

FIG. 7 shows a result of measuring a concentration of MCP-1 from blood collected 6 hours after administering lipid nanoparticles (LNP 126, 128, 218, 219, 220, 221, 284, 285, 286, 287, 297, 298, 299, 300) that include mRNA encoding Renilla luciferase (R/L) into the ears of ICR mice via I.D.

Referring to FIG. 7, particularly in the case of LNP 287, the concentration of MCP-1 compared to LNP 126 increased, and thus its utility as an mRNA vaccine may be expected. In particular, while the ionizable lipid used in LNP 287 is mRNA with HA antigen encoded in the absence of octadecyl gallate, given that the levels of IgG1 and IgG2a were significantly low in the primary and secondary immunizations, it may be noticed that the introduction of octadecyl gallate enhanced the immune efficacy. In the case of LNP 298, which had a significant increase in the protein expression efficiency compared to LNP 126 and a slight decrease in MCP-1 concentration, it may have the potential as a therapeutic agent capable of delivering nucleic acid drugs appropriately while reducing side effects caused by immune responses. In the case of LNP 219 and LNP 300, the protein expression efficiency and concentration of MCP-1 were slightly increased or similar to those of LNP 126, and thus its utility as an mRNA vaccine may be expected.

FIG. 8 shows a result of measuring a concentration of MCP-1 from blood collected 6 hours after administering lipid nanoparticles (LNP 126, 222, 223, 288, 289, 301, 302, 316, 317, 318, 319, 320, 321, 343, 344, 347, 348) that include mRNA encoding Renilla luciferase (R/L) into the ears of ICR mice via I.D.

Referring to FIG. 8, particularly in the case of LNP 317, the protein expression efficiency was similar to that of LNP 126, but the concentration of MCP-1 was greatly increased, and thus its utility as an mRNA vaccine may be expected. In the case of LNP 347 which has a very low MCP-1 concentration with a significant increase in the protein expression efficiency compared to LNP 126, it may have the potential as a therapeutic agent capable of appropriately delivering nucleic acid drugs while reducing side effects caused by immune responses. In the case of LNP 223, the protein expression efficiency and concentration of MCP-1 were similar to those of LNP 126, and thus its utility as an mRNA vaccine may be expected.

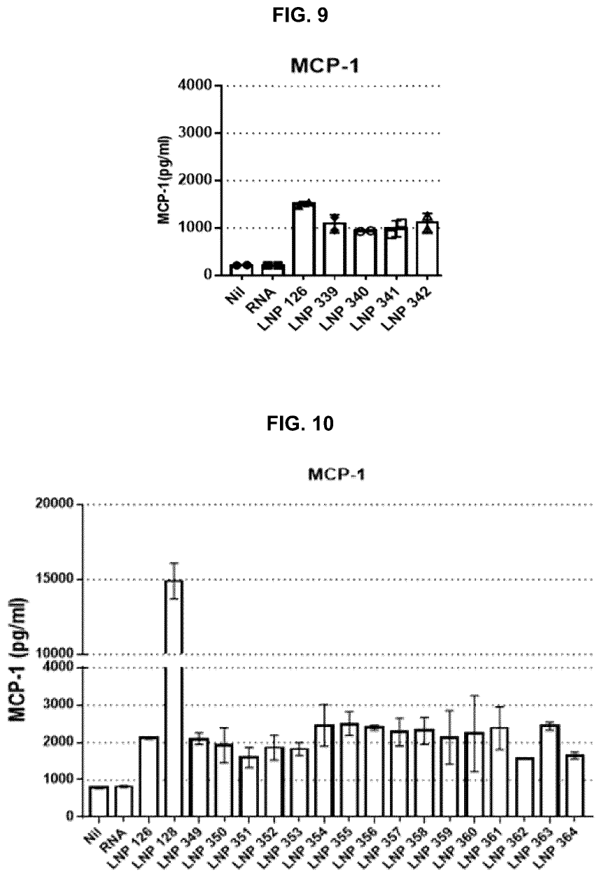

FIG. 9 shows a result of measuring a concentration of MCP-1 from blood collected 6 hours after administering lipid nanoparticles (LNP 126, 339, 340, 341, 342) that include mRNA encoding Renilla luciferase (R/L) into the ears of ICR mice via I.D.

Referring to FIG. 9, particularly in the case of LNP 342 which has a very low MCP-1 concentration with a very high protein expression efficiency compared to LNP 126, it may have the potential as a therapeutic agent capable of appropriately delivering nucleic acid drugs while reducing side effects caused by immune responses. In the case of LNP 339, LNP 340, and LNP 342 also, the protein expression efficiency was similar or higher than that of LNP 126 with a low MCP-1 concentration, such that it may have the potential as a therapeutic agent capable of appropriately delivering nucleic acid drugs while reducing side effects caused by immune responses.

FIG. 10 shows a result of measuring a concentration of MCP-1 from blood collected 6 hours after administering lipid nanoparticles (LNP 126, 128, 349, 350, 351, 352, 353, 354, 355, 356, 357, 358, 359, 360, 361, 362, 363, 364) that include mRNA encoding Renilla luciferase (R/L) into the ears of ICR mice via I.D.

Referring to FIG. 10, in the case of LNP 360 which has a low MCP-1 concentration with a very high protein expression efficiency compared to LNP 128, it may have the potential as a therapeutic agent capable of appropriately delivering nucleic acid drugs while reducing side effects caused by immune responses. In the case of LNP 350, LNP 352, LNP 359, and LNP 362 also, the protein expression efficiency was similar or higher than that of LNP 128, with a low MCP-1 concentration, such that it may have the potential as a therapeutic agent capable of appropriately delivering nucleic acid drugs while reducing side effects caused by immune responses.

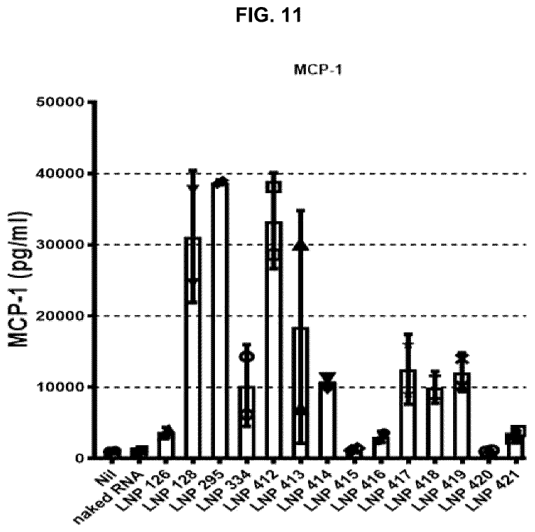

FIG. 11 shows a result of measuring a concentration of MCP-1 from blood collected 6 hours after administering lipid nanoparticles (LNP 126, 128, 295, 334, 412, 413, 414, 415, 416, 417, 418, 419, 420, 421) that include mRNA encoding Renilla luciferase (R/L) into the ears of ICR mice via I.D.

Referring to FIG. 11, in the case of LNP 414, LNP 419, and LNP 421 which has a low MCP-1 concentration with a very high protein expression efficiency compared to LNP 128, it may have the potential as a therapeutic agent capable of appropriately delivering nucleic acid drugs while reducing side effects caused by immune responses.

As described above, since a specific part of the content of the present disclosure is described in detail, for those of ordinary skill in the art, it is clear that the specific description is only a preferred embodiment, and the scope of the present disclosure is not limited thereby.

Thus, the substantial scope of the present disclosure will be defined by the appended claims and their equivalents.

1. A method of delivering a nucleic acid to a cell in a subject, comprising:
administering a lipid nanoparticle composition comprising a compound represented by the following Chemical Formula 1, a derivative thereof, or a pharmaceutically acceptable salt thereof as an active ingredient and the nucleic acid to the subject: 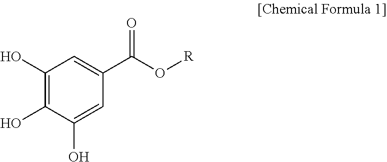
wherein, in the Chemical Formula 1,
R is a linear, saturated or unsaturated hydrocarbon with 3 to 22 carbon atoms.

2. The method of claim 1, wherein the compound comprises any one or more selected from the group consisting of compounds represented by the following Chemical Formulas 2 to 6:

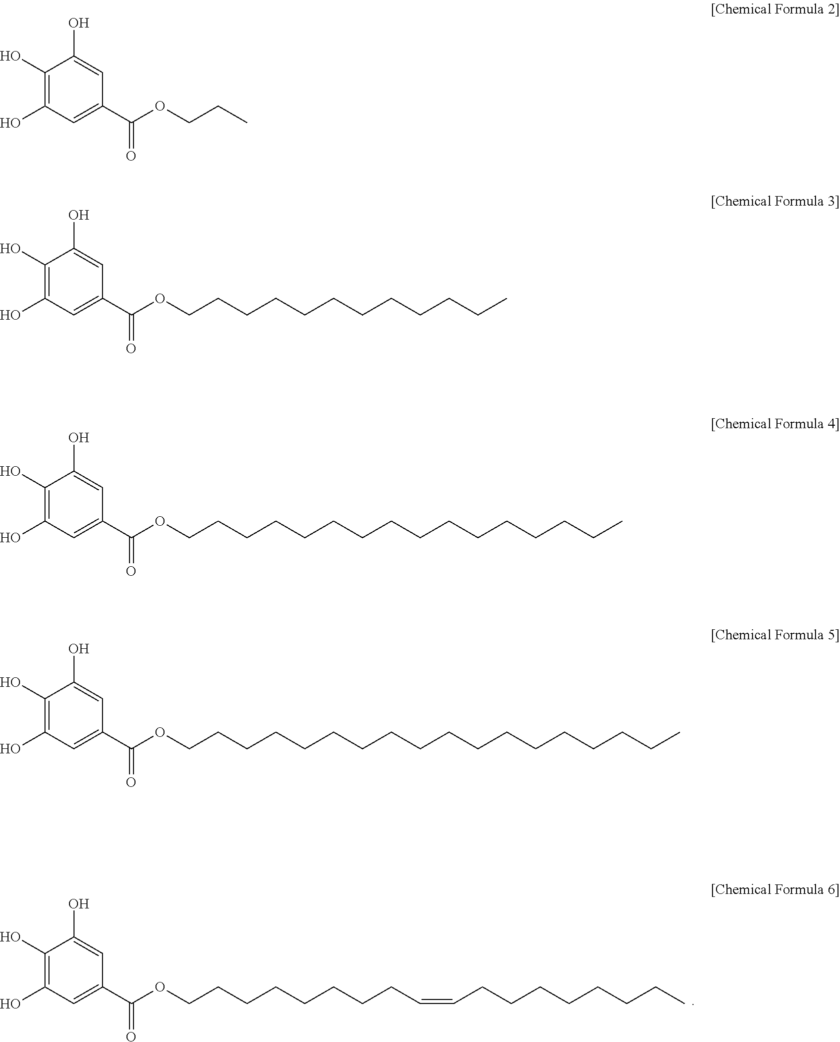 [SMILES: CCCOC(=O)c1cc(O)c(O)c(O)c1 ; CCCCCCCCCCCCOC(=O)c1cc(O)c(O)c(O)c1 ; CCCCCCCCCCCCCCCCOC(=O)c1cc(O)c(O)c(O)c1 ; CCCCCCCCCCCCCCCCCCOC(=O)c1cc(O)c(O)c(O)c1 ; CCCCCCCC/C=C\CCCCCCCCOC(=O)c1cc(O)c(O)c(O)c1]

3. The method of claim 1, wherein the compound is a helper lipid or a structure-maintaining lipid.

4. The method of claim 1, wherein the composition further comprises any one or more selected from the group consisting of an ionizable lipid, a helper lipid, a structure-maintaining lipid, a polyethylene glycol (PEG)-modified lipid, and an additive.

5. (canceled)

6. The method of claim 4, wherein the ionizable lipid is any one or more selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraene-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinoleyloxy-N,N-dimethylaminopropane (DODMA), 2-({8-[(30)-cholest-5-en-3-yloxy]octyl}oxy)-N,N-dimethyl-3-[(9Z,12Z)-octadeca-9,12-diene-1-yloxy]propane-1-amine (octyl-CLinDMA), (2R)-2-({8-[(33)-cholest-5-en-3-yloxy]octyl}oxy)-N,N-dimethyl-3-[(9Z,12Z)-octadeca-9,12-dien-1-yloxy]propan-1-amine (octyl-CLinDMA (2R)), (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (octyl-CLinDMA (2S)), 9-heptadecanyl 8-{(2-hydroxyethyl)[6-oxo-6-(undecyloxy)hexyl]amino}octanoate (SM-102), [(4-hydroxybutyl)azanediyl]di(hexane-6,1-diyl) bis(2-hexyldecanoate) (ALC-0315), and a pantothenic acid derivative.

7. The method of claim 6, wherein the pantothenic acid derivative is a compound selected from a compound represented by the following Chemical Formula 7, a stereoisomer thereof, a racemate thereof, or a pharmaceutically acceptable salt thereof: 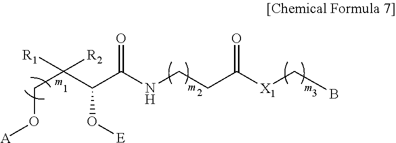
wherein, in the Chemical Formula 7,
R₁ and R₂ are the same or different and each independently hydrogen or hydrocarbons with 1 to 2 carbon atoms,
X₁ is O, NH, or S,
m₁, m₂, and m₃ are the same or different and each independently integers of 1 to 3, and
A is hydrogen or a compound represented by the following Chemical Formula 7-1, 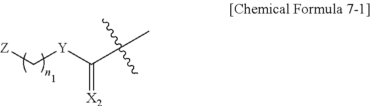
wherein, in the Chemical Formula 7-1,
Z is NR¹R²; a heterocyclic or aromatic compound with 3 to 8 carbon atoms; or a linear or branched, saturated or unsaturated hydrocarbon with 6 to 22 carbon atoms, wherein the R¹ and R² are the same or different and each independently linear or branched, saturated or unsaturated hydrocarbons with 1 to 6 carbon atoms; the heterocyclic or aromatic ring is substituted or unsubstituted with (C1˜C4)alkyl or di(C1˜C4)alkylamino(C1˜2)alkyl, and the hydrocarbon includes or does not include ester, ether, amide, carbamate, carbonate, or disulfide bonds,
X₂ is O or S,
Y is CH₂, NH, or O,
n₁ is an integer from 0 to 3, and
B is a compound represented by the following Chemical Formula 7-2 or NR^{1′}R^{2′}, and the R^{1′} and R^{2′} are the same or different and each independently linear or branched, saturated or unsaturated hydrocarbons with 1 to 6 carbon atoms, wherein the hydrocarbon includes or does not include ester, ether, amide, carbamate, carbonate, or disulfide bonds, and R^{1′} and R^{2′} are connected to each other to form a heterocyclic or aromatic ring with 3 to 8 carbon atoms, 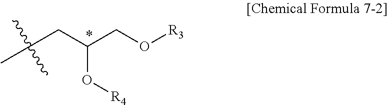
wherein, in the Chemical Formula 7-2,
R₃ and R₄ are the same or different and each independently linear or branched, saturated or unsaturated hydrocarbons with 6 to 22 carbon atoms, wherein the hydrocarbon includes or does not include ester, ether, amide, carbamate, carbonate, or disulfide bonds, and
E is hydrogen or a compound represented by the following Chemical Formula 7-3, and 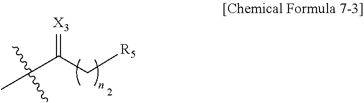
wherein, in the Chemical Formula 7-3,
X₃ is O or S,
n₂ is an integer of 0 to 3, and
R₅ is a linear or branched, saturated or unsaturated hydrocarbon with 6 to 22 carbon atoms, wherein the hydrocarbon includes or does not include ester, ether, amide, carbamate, carbonate, or disulfide bonds and is substituted or unsubstituted with thiolane or dithiolane.

8. The method of claim 7, wherein the pantothenic acid derivative comprises any one or more selected from the group consisting of compounds represented by the following Chemical Formulas 8 to 13:

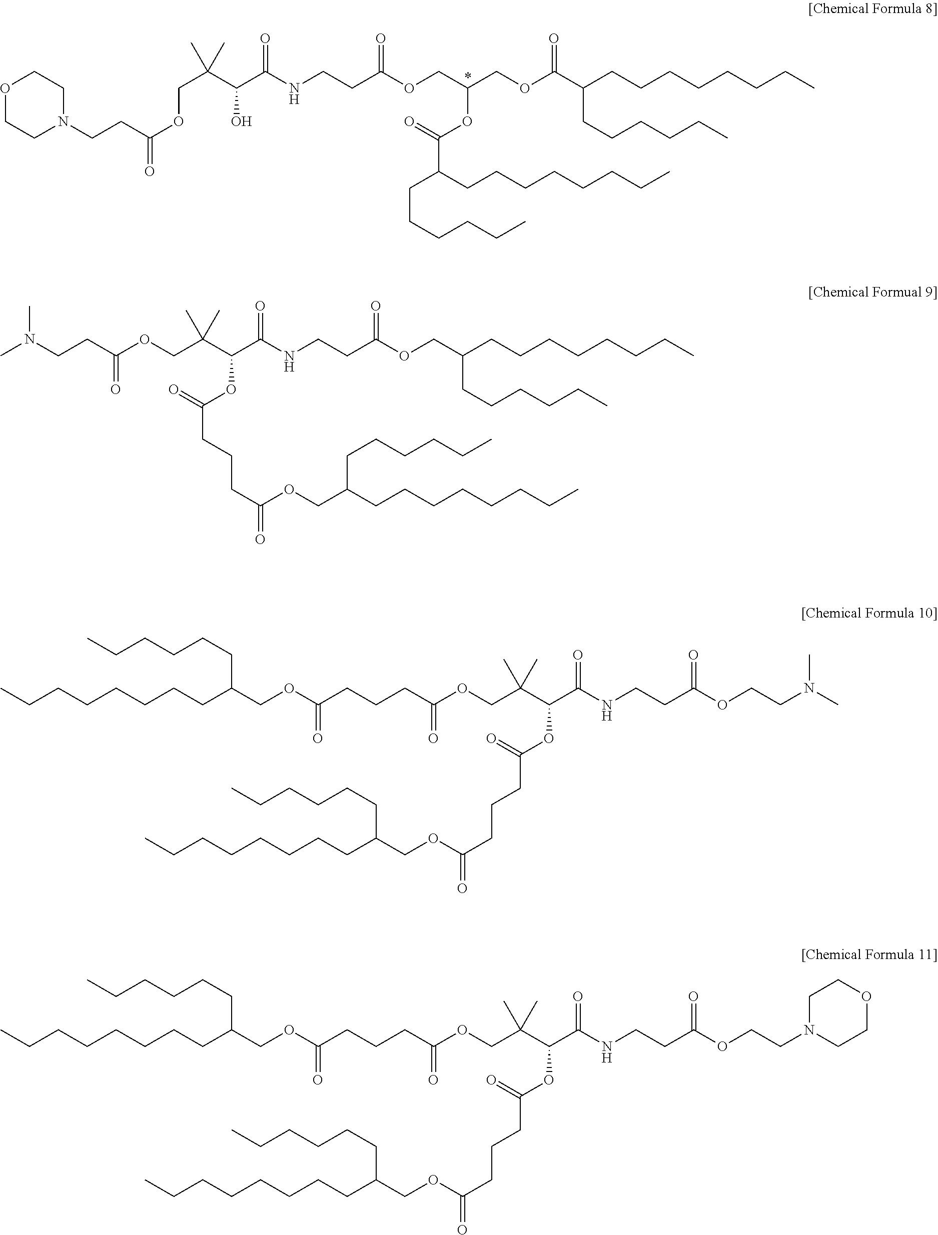

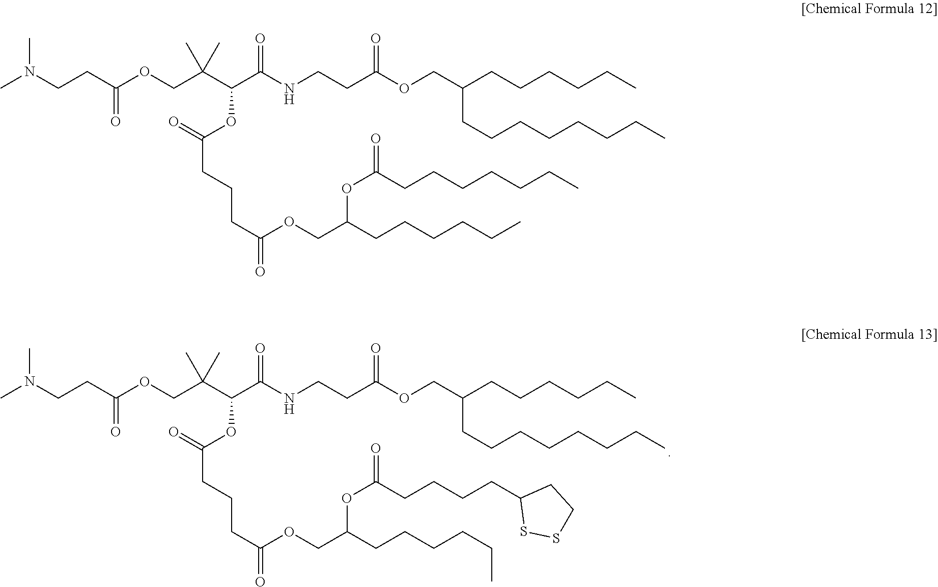

9. The method of claim 4, wherein, the helper lipid is any one or more selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinoleoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinoleoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl-phosphatidyl-ethanolamine (DPPE), 1,2-dimyristoyl-sn-glycero-3-phosphoethanolamine (DMPE), 1-stearoyl-2-oleoyl-phosphatidylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and a trehalose derivative.

10. The method of claim 9, wherein the trehalose derivative comprises a compound represented by the following Chemical Formula 14: 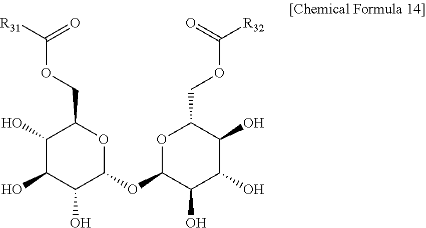
wherein, in the Chemical Formula 14,
R₃₁ and R₃₂ are the same or different and each independently linear or branched, saturated or unsaturated hydrocarbons with 6 to 22 carbon atoms.

11. The method of claim 4, wherein the polyethylene glycol (PEG)-modified lipid is selected from the group consisting of PEG-modified phosphatidylethanolamine, PEG-modified phosphatidic acid, PEG-modified ceramide, PEG-modified dialkylamine, PEG-modified diacylglycerol, PEG-modified dialkylglycerol, and a mixture thereof.

12. The method of claim 4, wherein the composition comprises 10 to 50 mol % of the compound, a derivative thereof, or a salt thereof, 20 to 60 mol % of the ionizable lipid, 5 to 20 mol % of the helper lipid, and 1 to 5 mol % of the PEG-modified lipid.

13. (canceled)

14. The method of claim 4, wherein the structure-maintaining lipid is any one or more selected from the group consisting of cholesterol, bile acid derivatives including butyl lithocholate, cholanic acid derivatives, lithocholic acid derivatives, flavonoids, vitamin A and derivatives thereof, vitamin E, vitamin K, coenzyme Q10, and beta-carotene.

15. A method of delivering a drug, comprising:
administering the lipid nanoparticle composition according to claim 1 and a therapeutic or prophylactic agent to a subject.

16. The method of claim 15, wherein the therapeutic or prophylactic agent is a vaccine or compound capable of inducing an immune response.

17. The method of claim 15, wherein the therapeutic or prophylactic agent is selected from the group consisting of small interfering RNA (siRNA), asymmetric interfering RNA (aiRNA), microRNA (miRNA), dicer-substrate RNA (dsRNA), small hairpin RNA (shRNA), messenger RNA (mRNA), and a mixture thereof.

18. A method of enhancing immune response in a subject, comprising:
administering an immunoenhancing composition comprising the lipid nanoparticle composition according to claim 1 to the subject.
